(19) United States
(12) Patent Application Publication
(10) Pub. No.: US 2024/0383865 A1
(43) Pub. Date: Nov. 21, 2024

(54) HIGH-ENERGY-VIOLET-ABSORBING VINYLIC MONOMERS

(71) Applicant: Alcon Inc., Fribourg (CH)

(72) Inventors: Troy Vernon Holland, Suwanee, GA (US); Adam K. Sniady, Lilburn, GA (US); Frank Chang, Cumming, GA (US)

(21) Appl. No.: 18/665,691

(22) Filed: May 16, 2024

Related U.S. Application Data
(60) Provisional application No. 63/503,035, filed on May 18, 2023.

(51) Int. Cl. C07D 249/20; B29D 11/00; B29K 55/00; B29K 105/00; C08F 220/54; C08F 230/08; C08K 5/00; G02B 1/04; G02C 7/10

(52) U.S. Cl. CPC C07D 249/20; B29D 11/00076; C08F 220/54; C08F 230/08; C08K 5/0091; G02B 1/043; G02C 7/104; B29K 2055/00; B29K 2105/0002; B29K 2105/0032; B29K 2995/0026; B29K 2995/0046; B29K 2995/0068

(57) ABSTRACT

Described herein is high-energy-violet-light (HEVL) absorbing benzotriazole vinylic monomers that have an adequate solubility in an organic solvent (e.g., 1-propanol), a better compatibility with polymerizable components present in a hydrogel lens formulation, and an absorption peak at a wavelength of from 410 nm to 425 nm. They are suitable for making hydrogel contact lenses that can significantly block HEVL from 380 nm to 450 nm but cannot block significantly blue lights (from 450 nm to 495 nm).

This application claims the benefit under 35 USC § 119(e) of U.S. Provisional appl. No. 63/503,035 filed 18 May 2023, herein incorporated by reference in its entirety.

This invention is related to benzotriazole vinylic monomers capable of absorbing high-energy-violet (HEVL) radiation (with wavelengths from 380 nm to 450 nm) and their uses for producing hydrogel contact lenses capable of blocking ultra-violet (“UV”) radiation and HEVL radiation.

### BACKGROUND

LED lights and electronic device, including smart phones, computer screens, LCD and LED televisions, have been extensively used. They typically can emit short wavelength visible light, including violet light (380-450 nm) and blue light (450-495 nm). Such short wavelength visible lights were shown to be damaging to cells both in in vitro and in vivo studies reported in Experimental Eye Research 2006, 83, 1493; J. Cataract Refrac Surg 2009, 35, 354; Graefe's Arch Clin Exp Ophthalmol 2008, 246, 671; Acta Ophthalmologica Scandinavica 2006, 84, 4; Br J Ophthalmol 2006, 90, 784; Optometry and Vision Science 2011, 88 (6), 1. A great effort has been made to develop HEVL-filtering ophthalmic lenses, such as, spectacles, contact lenses, intraocular lenses, etc. to protect eyes from increasing exposures of HEVL due to widely use of LED lights and LED displays, e.g., smart phone, TV and computer monitor (see, e.g., U.S. Pat. Nos. 4,612,358, 4,528,311, 4,716,234, 4,878,748, 5,400,175, 5,662,707, 6,158,862, 6,955,430, 7,556,376, 7,803,359, 8,153,703, 8,232,326, 8,360,574, 8,585,938, 8,882,267, 9,377,569, 9,683,102, 9,814,658, 10,551,637, and 10,610,472; U.S. Pat. Appl. Pub. Nos. 20170242274, 20180371139, 20190002415, 20190002459, 20190271798, 20190339544, 20200002267, 20200095187, 20200407324, and 20200407337).

TOTAL30® (from Alcon) is the first contact lens to offer HEVL-filtering capability that is constantly in effect while wearing the lenses regardless of the lighting conditions. TOTAL30® not only includes Class I UV absorption for protection against UVA and UVB rays (i.e., filtering more than 90% UVA and 99% UVB rays), but also can filter out approximately 33% of HEVL rays entering the eye (between 380-450 nm). Alcon subsequently launched a second product, TOTAL1®, which like TOTAL30® can block 90% UVA, 99% UVB, and 33% HEVL. Johnson & Johnson Vision Care recently also launched ACUVUE® OASYS MAX 1-DAY which can block up to 45% HEVL according to its published 510 (k) Premarket Notification (K210930).

It would still be desirable to have a contact lens product with HEVL-filtering capability much higher than currently available commercial contact lenses to better protect wears' eyes from HEVL damages, especially violet light damages. However, it is quite a challenging to find a suitable polymerizable HEVL-absorbing dye that can absorb a significant amount of HEVL light and have a good solubility in a lens-forming composition, a good compatibility with other polymerizable components in the lens-forming composition, and a good light fastness.

Therefore, there are still needs for new HEVL-absorbing vinylic monomers suitable for making hydrogel contact lenses, especially silicone hydrogel contact lenses, capable of significantly filtering HEVL and needs for such high HEVL-filtering hydrogel contact lenses.

### SUMMARY OF THE INVENTION

In one aspect, the invention provides a HEVL-absorbing vinylic monomer comprising a moiety of benzotriazole and a (meth)acryloyl group and having a HEVL absorption peak at a wavelength of from 410 nm and 425 nm and a solubility of at least 2.5 mg/mL in 1-propanol.

In another aspect, the invention provides a method for producing HEVL-absorbing hydrogel contact lenses from a lens formulation comprising a UV-absorbing vinylic monomer of the invention.

The invention provides in a further aspect hydrogel contact lenses comprising monomeric units of a HEVL-absorbing vinylic monomer of the invention.

### BRIEF DESCRIPTIONS OF THE DRAWINGS

FIG. 1 shows the procedures for synthesizing a HEVL-absorbing benzotriazole vinylic monomer of the invention according to a preferred embodiment.

FIG. 2 shows the procedures for synthesizing a HEVL-absorbing benzotriazole vinylic monomer of the invention according to a preferred embodiment.

FIG. 3 shows the UV/visible absorption spectra of dilute solutions of 2 HEVL-absorbing benzotriazole-containing vinylic monomers (UV28 and UV35) as measured in a 1 cm cuvette: dot line—40 ppm UV28; dashed line—40 ppm UV35.

FIG. 4 shows the UV/visible transmission spectra of UV/visible transmission spectra of contact lenses obtained from lens formulations 3-2 (dashed line) and 4-1 (dot line).

FIG. 5 shows the UV/visible absorption spectra of dilute solutions of 4 HEVL-absorbing benzotriazole-containing vinylic monomers (UV29, UV28, UV23, and UV40) in 1-propanol as measured in a 1 cm cuvette: dot line-40 ppm UV29; dashed line—40 ppm UV28; solid line—40 ppm UV23; dashed line-40 ppm UV40.

### DETAILED DESCRIPTION OF THE INVENTION

Unless defined otherwise, all technical and scientific terms used herein have the same meaning as commonly understood by one of ordinary skill in the art to which this invention belongs. Generally, the nomenclature used herein and the laboratory procedures are well known and commonly employed in the art. Conventional methods are used for these procedures, such as those provided in the art and various general references. Where a term is provided in the singular, the inventors also contemplate the plural of that term. The nomenclature used herein and the laboratory procedures described below are those well known and commonly employed in the art.

“About” as used herein means that a number referred to as “about” comprises the recited number plus or minus 1-10% of that recited number.

“Optional” or “optionally” means that the subsequently described event or circumstance can or cannot occur, and that the description includes instances where the event or circumstance occurs and instances where it does not.

An “ophthalmic device”, as used herein, refers to a contact lens (hard or soft), an intraocular lens, a corneal onlay, other ophthalmic devices (e.g., stents, glaucoma shunt, or the like) used on or about the eye or ocular vicinity.

“Contact Lens” refers to a structure that can be placed on or within a wearer's eye. A contact lens can correct, improve, or alter a user's eyesight, but that need not be the case. A contact lens can be made of any appropriate material known in the art or later developed, and can be a soft lens, a hard lens, or a hybrid lens.

A “hydrogel contact lens” refers to a contact lens comprising a hydrogel bulk (core) material. A hydrogel bulk material can be a non-silicone hydrogel material or preferably a silicone hydrogel material.

A “hydrogel” or “hydrogel material” refers to a crosslinked polymeric material which has three-dimensional polymer networks (i.e., polymer matrix), is insoluble in water, but can hold at least 10% by weight of water in its polymer matrix when it is fully hydrated (or equilibrated).

A siloxane, which often also described as a silicone, refers to a molecule having at least one moiety of —Si—O—Si— where each Si atom carries two organic groups as substituents.

A “silicone hydrogel” or “SiHy” refers to a silicone-containing hydrogel obtained by copolymerization of a polymerizable composition comprising at least one silicone-containing monomer or at least one silicone-containing macromer or at least one crosslinkable silicone-containing prepolymer.

As used in this application, the term “non-silicone hydrogel” refers to a hydrogel that is theoretically free of silicon.

“Hydrophilic,” as used herein, describes a material or portion thereof that will more readily associate with water than with lipids.

The term “room temperature” refers to a temperature of about 22° C. to about 26° C. The term “soluble”, in reference to a compound or material in a solvent, means that the compound or material can be dissolved in the solvent to give a solution with a concentration of at least about 0.5% by weight at room temperature (i.e., a temperature of about 22° C. to about 26° C.).

The term “insoluble”, in reference to a compound or material in a solvent, means that the compound or material can be dissolved in the solvent to give a solution with a concentration of less than 0.01% by weight at room temperature (as defined above).

A “vinylic monomer” refers to a compound that has one sole ethylenically unsaturated group, is soluble in a solvent, and can be polymerized actinically or thermally.

The term “ethylenically unsaturated group” is employed herein in a broad sense and is intended to encompass any groups containing at least one >C═CH₂ group. Exemplary ethylenically unsaturated groups include without limitation (meth)acryloyl

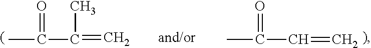

vinyloxycarbonylamino

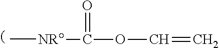

in which R^{o} is H or C₁-C₄ alkyl), vinyloxycarbonyloxy

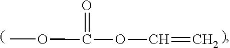

allyl, vinyl, styrenyl, or other C═C containing groups.

As used herein, “actinically” in reference to curing, crosslinking or polymerizing of a polymerizable composition, a prepolymer or a material means that the curing (e.g., crosslinked and/or polymerized) is performed by actinic irradiation, e.g., UV/visible light irradiation, or the like. Thermal curing or actinic curing methods are well-known to a person skilled in the art.

An “acrylic monomer” refers to a vinylic monomer having one sole (meth)acryloyl group. Examples of acrylic monomrs includes (meth)acryloxy [or (meth)acryloyloxy] monomers and (meth)acrylamido monomers.

An “(meth)acryloxy monomer” or “(meth)acryloyloxy monomer” refers to a vinylic monomer having one sole group of

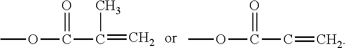

An “(meth)acrylamido monomer” refers to a vinylic monomer having one sole group of

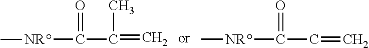

in which R^{o} is H or C₁-C₄ alkyl.

The term “(meth)acrylamide” refers to methacrylamide and/or acrylamide.

The term “(meth)acrylate” refers to methacrylate and/or acrylate.

An “N-vinyl amide monomer” refers to an amide compound having a vinyl group (—CH═CH₂) that is directly attached to the nitrogen atom of the amide group.

The term “ene group” refers to a monovalent radical of CH₂═CH— or CH₂═CCH₃— that is not covalently attached to an oxygen or nitrogen atom or a carbonyl group.

An “ene monomer” refers to a vinylic monomer having one sole ene group.

A “vinyloxycarbonylamino monomer” refers to a vinylic monomer having one sole vinyloxycarbonylamino group.

A “vinylaminocarbonyloxy monomer” refers to a vinylic monomer having one sole vinylaminocarbonyloxy group.

A “vinylaminocarbonylamino monomer” refers to a vinylic monomer having one sole vinylaminocarbonylamino group.

A “hydrophilic vinylic monomer” refers to a vinylic monomer which typically yields a homopolymer that is water-soluble or can absorb at least 10 percent by weight of water.

A “hydrophobic vinylic monomer” refers to a vinylic monomer which typically yields a homopolymer that is insoluble in water and can absorb less than 10% by weight of water.

As used in this application, the term “vinylic crosslinker” refers to an organic compound having at least two ethylenically unsaturated groups. A “vinylic crosslinking agent” refers to a vinylic crosslinker having a molecular weight of 700 Daltons or less.

As used in this application, the term “polymer” means a material formed by polymerizing/crosslinking one or more monomers or macromers or prepolymers or combinations thereof.

A “macromer” or “prepolymer” refers to a compound or polymer that contains multiple ethylenically unsaturated groups and has a number average molecular weight of greater than 700 Daltons.

As used in this application, the term “molecular weight” of a polymeric material (including monomeric or macromeric materials) refers to the number-average molecular weight unless otherwise specifically noted or unless testing conditions indicate otherwise. A skilled person knows how to determine the molecular weight of a polymer according to known methods, e.g., GPC (gel permeation chromatography) with one or more of a refractive index detector, a low-angle laser light scattering detector, a multi-angle laser light scattering detector, a differential viscometry detector, a UV detector, and an infrared (IR) detector; MALDI-TOF MS (matrix-assisted laser desorption/ionization time-of-flight mass spectroscopy); ¹H NMR (Proton nuclear magnetic resonance) spectroscopy, etc.

A “polysiloxane segment” or “polydiorganosiloxane segment” interchangeably refers to a polymer chain segment (i.e., a divalent radical) of

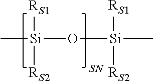

in which SN is an integer of 3or larger and each of R_{S1} and R_{S2} independent of one another are selected from the group consisting of: C₁-C₁₀ alkyl; phenyl; C₁-C₄-alkyl-substituted phenyl; C₁-C₄-alkoxy-substituted phenyl; phenyl-C₁-C₆-alkyl; C₁-C₁₀ fluoroalkyl; C₁-C₁₀ fluoroether; aryl; aryl C₁-C₁₈ alkyl; -alk-(OC₂H₄)_{γ1}—OR^{o} (in which alk is C₁-C₆ alkylene diradical, R^{o} is H or C₁-C₄ alkyl and γ1 is an integer from 1 to 10); a C₂-C₄₀ organic radical having at least one functional group selected from the group consisting of hydroxyl group (—OH), carboxyl group (—COOH), amino group (—NR_{N1}R_{N1}′), amino linkages of —NR_{N1}—, amide linkages of —CONR_{N1}—, amide of —CONR_{N1}R_{N1}′, urethane linkages of —OCONH—, and C₁-C₄ alkoxy group, or a linear hydrophilic polymer chain, in which R_{N1} and R_{N1}′ independent of each other are hydrogen or a C₁-C₁₅ alkyl; and a photochromic organic radical having a photochromic group.

A “polydiorganosiloxane vinylic monomer” or “polysiloxane vinylic monomer” interchangeably refers to a compound comprising at least one polysiloxane segment and one sole ethylenically-unsaturated groups.

A “polydiorganosiloxane vinylic crosslinker” or “polysiloxane vinylic crosslinker” interchangeably refers to a compound comprising at least one polysiloxane segment and at least two ethylenically-unsaturated groups.

The term “fluid” as used herein indicates that a material is capable of flowing like a liquid.

As used in this application, the term “clear” in reference to a polymerizable composition means that the polymerizable composition is a transparent solution or liquid mixture having a light transmissibility of 85% or greater (preferably 90% or greater) in the range between 400 to 700 nm.

A free radical initiator can be either a photoinitiator or a thermal initiator. A “photoinitiator” refers to a chemical that initiates free radical crosslinking/polymerizing reaction by the use of light. A “thermal initiator” or “thermal free radical initiator” interchangeably refers to a chemical that initiates free radical crosslinking/polymerizing reaction by the use of heat energy.

The term “monovalent radical” and “monovalent group” interchangeably refer to an organic radical or group that is obtained by removing a hydrogen atom from an organic compound and that forms one bond with one other group in an organic compound. Examples include without limitation, alkyl (by removal of a hydrogen atom from an alkane), alkoxy (or alkoxyl) (by removal of one hydrogen atom from the hydroxyl group of an alkyl alcohol), thiyl (by removal of one hydrogen atom from the thiol group of an alkylthiol), cycloalkyl (by removal of a hydrogen atom from a cycloalkane), cycloheteroalkyl (by removal of a hydrogen atom from a cycloheteroalkane), aryl (by removal of a hydrogen atom from an aromatic ring of the aromatic hydrocarbon), heteroaryl (by removal of a hydrogen atom from any ring atom), amino (by removal of one hydrogel atom from an amine), etc.

The term “divalent radical” and “divalent group” interchangeably refer to an organic radical or group that is obtained by removing two hydrogen atoms from an organic compound and that forms two bonds with other two groups in an organic compound. For example, an alkylene divalent radical (i.e., alkylenyl) is obtained by removal of two hydrogen atoms from an alkane, a cycloalkylene divalent radical (i.e., cycloalkylenyl) is obtained by removal of two hydrogen atoms from the cyclic ring.

In this application, the term “substituted” in reference to an alkyl or an alkylenyl means that the alkyl or the alkylenyl comprises at least one substituent which replaces one hydrogen atom of the alkyl or the alkylenyl and is selected from the group consisting of hydroxyl (—OH), carboxyl (—COOH), —NH₂, sulfhydryl (—SH), C₁-C₄ alkyl, C₁-C₄ alkoxy, C₁-C₄ alkylthio (alkyl sulfide), C₁-C₄ acylamino, C₁-C₄ alkylamino, di-C₁-C₄ alkylamino, and combinations thereof.

The term “silicone hydrogel lens formulation” or “SiHy lens formulation” interchangeably refers to a polymerizable composition that comprises all necessary polymerizable components for producing a SiHy contact lens or a SiHy lens bulk material as well known to a skilled person.

A “UV-absorbing vinylic monomer” refers to a compound comprising one sole ethylenically-unsaturated group and can absorb predominantly UV lights between 280 nm to 380 nm. It is understood that a UV-absorbing vinylic monomer does not absorb or absorbs negligibly lights having a wavelength greater 400 nm (i.e., having a % T at 400 nm of greater than 90% when tested with a solution of the UV-absorbing vinylic monomer at a concentration of 0.1 mM and a path length of 1 cm).

A “HEVL-absorbing vinylic monomer” refers to a compound comprising one sole ethylenically-unsaturated group and can absorb HEVL between 380 nm and 450 nm. It is understood that a HEVL-absorbing vinyli monomer can also absorb UV lights between 280 nm and 380 nm.

“UVA” refers to radiation occurring at wavelengths between 315 and 380 nanometers; “UVB” refers to radiation occurring between 280 and 315 nanometers; “HEVL” refers to radiation occurring at wavelengths between 380 and 450 nanometers.

“UVA transmittance” (or “UVA % T”), “UVB transmittance” or “UVB % T”, and “HEVL-transmittance” or “HEVL % T” are calculated by the following formula.

$$UVA \% T = Average \% Transmission between 315 nm and 380 nm \times 100$$

$$UVB \% T = Average \% Transmission between 280 nm and 315 nm \times 100$$

$$HEVL \% T = Average \% Transmission between 380 nm and 450 nm \times 100$$

$$HEVL \% filtration = 100 \% - HEVL \% T$$

“% T at a wavelength” refers to a percent transmission at the specified wavelength.

In general, the invention is directed to a class of HEVL-absorbing benzotriazole vinylic monomers which have an adequate solubility (i.e., ≥2.5 mg/mL) in an organic solvent (e.g., 1-propanol), a better compatibility with polymerizable components present in a hydrogel lens formulation (especially in a silicone hydrogel lens formulation), and an absorption peak at a wavelength of from 410 nm to 425 nm. A HEVL-absorbing benzotriazole vinylic monomer of the invention is suitable for making hydrogel contact lenses capable of significantly filtering HEVL. Further, it is believed that by having an absorption peak at a wavelength of from 410 nm to 425 nm (more preferably from 410 nm to 422 nm, even more preferably from 412 nm to 420 nm), a hydrogel contact lens containing one or two HEVL-absorbing vinylic monomers of the invention would not block significantly blue lights (from 450 nm to 495 nm) and subsequently would provide a minimally-altered color perception, a minimally reduced color vision, a minimized reduction in scotopic visual function, a minimally reduced contrast sensitivity in mesopic and scotopic conditions, and a minimized disruption of circadian photo-entrainment.

In one aspect, the present invention provides a HEVL-absorbing benzotriazole vinylic monomer of formula (I)

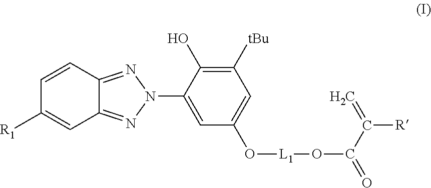

in which tBu is tert-butyl group, R′ is H or CH₃, R₁ is Cl or CF₃, L₁ is a divalent C₅-C₆ alkylene group or a divalent group of —C₂H₄—(OC₂H₄)ₙ— in which n is 1, 2 or 3.

In accordance with this aspect of the invention, the linkage L₁ has an extended spacer and as such can have a higher solubility in 1-propanol (compared to one with a shorter L₁, e.g., L₁═CH₂CH₂CH₂). Further, R₁ is an organic group having a Hammett substituent constant (σₚ) of about 0.23 (R₁═Cl) or about 0.54 (R₁═CF₃). It is believed that the electron withdrawing capability (Hammett substituent constant, σₚ) of a substituent on the aromatic ring of a benzotriazole can correlate with the wavelength of the absorption peak. The more the electron withdrawing character (i.e., larger σₚ), the more the red shift of the absorption peak. When R₁ is Cl or CF₃, the monomer can have an absorption peak at around 364 nm or around 368 nm.

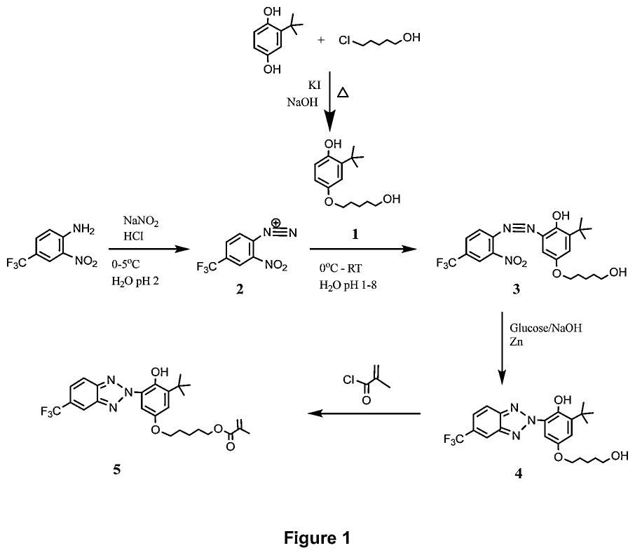

Benzotriazole vinylic monomers of formula (I) can be prepared using methods described in U.S. Pat. Nos. 4,716,234, 8,153,703, and 8,585,938. For example, a benzotriazole vinylic monomer of formula (II) in which R′═CH₃, R₁═CF₃, and L₁═C₅H₁₀ can be synthesized according to the procedures shown in FIG. 1. This synthetic pathway starts with the preparation of 2-tert-butyl-4-(5′-hydroxy-pentyloxy) phenol (“Compound 1”) by reacting 5-chloro-1-pentanol with tert-butylhydroquinone as described in U.S. Pat. No. 4,716,234. Then, 4-trifluoromethyl-2-nitroaniline is converted to the diazonium salt (“Compound 2”) which is azo coupled with Compound 1 followed by reduction of nitro azo intermediate (“Compound 3”) with glucose and zinc powder, which closes the benzotriazole ring yielding a benzotriazole compound (“Compound 4”), according to the procedures described in U.S. Pat. No. 8,585,938. In the final step, Compound 4 is reacted with methacryloyl chloride to obtain 2-[2′hydroxy-3′-tert-butyl-5′(5″-methacryloyloxy)pentyloxyphenyl]-5-trifluoromethyl-2H-benzotriazole (“Compound 5”), according to the procedures described in U.S. Pat. No. 4,716,234.

It is understood that in the first step in Scheme I, one can substitute 5-chloro-1-pentanol with 6-chloro-1-hexanol, hydroxyethoxyethyl chloride (ClC₂H₄OC₂H₄OH), hydroxyethoxy-ethoxyethyl chloride (ClC₂H₄OC₂H₄OC₂H₄OH), or hydroxyethoxyethoxyethoxyethyl chloride (ClC₂H₄OC₂H₄OC₂H₄OC₂H₄OH) to obtain a benzotriazole vinylic monomer of formula (I) in which L₁ is one of the other recited divalent radicals (groups).

In another aspect, the present invention provides a HEVL-absorbing benzotriazole vinylic monomer of formula (II)

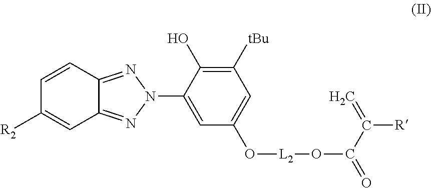

in which tBu is tert-butyl group, R′ is H or CH₃, R₂ is an organic group which is free of any halogen atom and has a Hammett substituent constant (σₚ) of from about 0.15 to about 0.70(preferably from about 0.20 to about 0.65), L₂ is a divalent C₂-C₁₀ alkylene group or a divalent group of —C₂H₄—(OC₂H₄)ₙ— in which n is 1, 2 or 3.

In formula (II), R₂ preferably is —CH₂CN (σₚ˜0.18), —OCOCH₃ (σₚ˜0.31), —CO(CH₃)₃ (σₚ˜0.32), —SO₃⁻ (σₚ˜0.35), —CONH₂ (σₚ˜0.36), —OSO₂CH₃ (σₚ˜0.36), —CONHCH₃ (σₚ˜0.36), —CON(CH₃)₂ (σₚ˜0.36), —CH₂N⁺(CH₃)₃ (σₚ˜0.44), —COOH (σₚ˜0.45), —COOCH₃ (σₚ˜0.45), —COOC₂H₅ (σₚ˜0.45), —COC₂H₅ (σₚ˜0.48), —COCH₃ (σₚ˜0.50), or —CN (σₚ˜0.66).

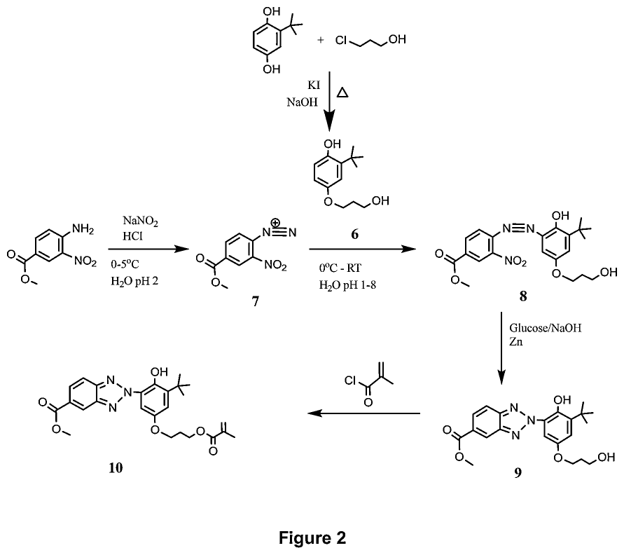

Benzotriazole vinylic monomers of formula (II) can be prepared according to the procedures similar to Scheme II. For example, a benzotriazole vinylic monomer of formula (II) in which R′═CH₃, R₂═COOCH₃, and L₂═C₃H₆ can be synthesized according to the procedures sown in FIG. 2. This synthetic pathway starts with the preparation of 2-tert-butyl-4-(5′-hydroxy-propoxy) phenol (“Compound 6”) by reacting 3-chloro-1-propanol with tert-butylhydroquinone. Then, methyl 4-amino-2-nitrobenzoate is converted to the diazonium salt (“Compound 7”) which is azo coupled with Compound 6 followed by reduction of nitro azo intermediate (“Compound 8”) with glucose and zinc powder, which closes the benzotriazole ring yielding a benzotriazole compound (“Compound 9”). In the final step, Compound 9 is reacted with methacryloyl chloride to obtain methyl 2-(3-(tert-butyl)-2-hydroxy-5-(3-(methacryloyloxy)propoxy) phenyl)-2H-benzotriazole-5-carboxylate (“Compound 10”).

It is understood that other benzotriazole vinylic monomers of formula (II) can be prepared from tert-butylhydroquinone, a chloro-substituted alkyl alcohol (e.g., ClC₄H₈OH, ClC₅H₁₀OH, ClC₆H₁₂OH, ClC₇H₁₄OH, ClC₈H₁₆OH, ClC₂H₄OC₂H₄OH, ClC₂H₄OC₂H₄OC₂H₄OH, ClC₂H₄OC₂H₄OC₂H₄OC₂H₄OH, etc.), a 2-nitroaniline substituted at position 4 with a substituent (e.g., —CH₂CN, —OCOCH₃, —CO(CH₃)₃, —SO₃, —CONH₂, —OSO₂CH₃, —CONHCH₃, —CON(CH₃)₂, —CH₂N⁺(CH₃)3, —COOH, —COOC₂H₅, —COC₂H₅, —COCH₃, or —CN) according to the procedures in Scheme II.

A benzotriazole vinylic monomer of the invention described above can find particular uses in making UV-absorbing ophthalmic devices, preferably intraocular lenses and hydrogel contact lenses, more preferably hydrogel contact lenses.

In a further aspect, the present invention provides a method for making HEVL-filtering hydrogel contact lenses, comprising the steps of: (1) obtaining a polymerizable composition comprising (a) (from about 0.1% to about 2.5% by weight of, preferably from about 0.2% to about 2.0% by weight of, more preferably from about 0.3% to about 1.8% by weight of, even more preferably from about 0.4% to about 1.6% by weight of) at least one UV-absorbing vinylic monomer, (b) (from about 0.2% to about 3.0% by weight of, preferably from about 0.25% to about 2.5% by weight of, more preferably from about 0.3% to about 2.5% by weight of, even more preferably from about 0.35% to about 2.25% by weight of) at least one HEVL-absorbing benzotriazole vinylic monomer of formula (I) or (II) (as defined above), (c) (from about 0.1% to about 2.0% by weight of, preferably from about 0.25% to about 1.75% by weight of, more preferably from about 0.5% to about 1.5% by weight of, even more preferably from about 0.75% to about 1.25% by weight of) at least free-radical initiator, (d) at least one blue-tinting agent which is Cu(II)-phthalocyanine blue pigment particles or at least one polymerizable blue dye, and (e) at least one polymerizable components selected from the group consisting of a hydrophilic vinylic monomer, a siloxane-containing vinylic monomer, a polysiloxane vinylic crosslinker, a non-silicone vinylic crosslinker, a non-silicone hydrophobic vinylic monomer, and combinations thereof; (2) introducing the polymerizable composition into a mold for making a hydrogel contact lens, wherein the mold has a first mold half with a first molding surface defining the anterior surface of the contact lens and a second mold half with a second molding surface defining the posterior surface of the contact lens, wherein said first and second mold halves are configured to receive each other such that a cavity is formed between said first and second molding surfaces; and (3) curing thermally or actinically the lens formulation in the mold to form the HEVL-filtering hydrogel contact lens, wherein the formed HEVL-filtering hydrogel contact lens has a UVA % T of less than 5%, UVB%T of about 1% or less, a HEVL % T of about 35% or less (preferably about 32% or less, more preferably about 30% or less, even more preferably about 28% or less).

In accordance with the invention, any UV-absorbing vinylic monomers can be used in the invention, so long as they can provide Class I UV protection in combination with one or more HEVL-absorbing vinylic monomers of the invention. Preferably, at least one UV-absorbing vinylic monomer can be one or more benzotriazole-containing vinylic monomers (i.e., ones each having a benzotriazole-moiety) selected from the group consisting of 2-(2′-hydroxy-5′-vinyl-phenyl)-2H-benzotriazole, 2-(2′-hydroxy-5′-methacryloxyphenyl)-2H-benzotriazole, 2-(2′-hydroxy-5′-acryloyloxyphenyl)-2H-benzotriazole, 2-[2′-hydroxy-5′-(2-methacryloxyethyl)phenyl)]-2H-benzotriazole (Norbloc), 2-[2′-hydroxy-5′-(2-acryloxyethyl)phenyl)]-2H-benzotriazole, 2-(2′-hydroxy-5′-methacryloxypropyl-phenyl)-2H-benzotriazole, 2-(2′-hydroxy-5′-acryloxypropyl-phenyl)-2H-benzotriazole, and combinations thereof. More preferably, at least one UV-absorbing vinylic monomer is 2-[2′-hydroxy-5′-(2-methacryloxyethyl)phenyl)]-2H-benzotriazole.

In accordance with the invention, a free-radical initiator can be a free-radical thermal initiator or a free-radical photoinitiator.

In accordance with the invention, any thermal free-radical initiators can be used in the invention. Suitable thermal free-radical initiators are known to a skilled artisan and include, for example, peroxides, hydroperoxides, azo-bis(alkyl- or cycloalkylnitriles), persulfates, percarbonates, or mixtures thereof. Examples of preferred thermal free-radical initiators include without limitation benzoyl peroxide, t-butyl peroxide, t-amyl peroxybenzoate, 2,2-bis(tert-butylperoxy)butane, 1,1-bis(tert-butylperoxy)cyclohexane, 2,5-Bis(tert-butylperoxy)-2,5-dimethylhexane, 2,5-bis(tert-butylperoxy)-2,5-dimethyl-3-hexyne, bis(1-(tert-butylperoxy)-1-methylethyl) benzene, 1,1-bis(tert-butylperoxy)-3,3,5-trimethylcyclohexane, di-t-butyl-diperoxy-phthalate, t-butyl hydroperoxide, t-butyl peracetate, t-butyl peroxybenzoate, t-butylperoxy isopropyl carbonate, acetyl peroxide, lauroyl peroxide, decanoyl peroxide, dicetyl peroxydicarbonate, di(4-t-butylcyclohexyl)peroxy dicarbonate (Perkadox 16S), di(2-ethylhexyl)peroxy dicarbonate, t-butylperoxy pivalate (Lupersol 11); t-butylperoxy-2-ethylhexanoate (Trigonox 21-C50), 2,4-pentanedione peroxide, dicumyl peroxide, peracetic acid, potassium persulfate, sodium persulfate, ammonium persulfate, 2,2′-azobis(4-methoxy-2,4-dimethylvaleronitrile) (VAZO 33), 2,2′-Azobis[2-(2-imidazolin-2-yl)propane] dihydrochloride (VAZO 44), 2,2′-azobis(2-amidinopropane) dihydrochloride (VAZO 50), 2,2′-azobis(2,4-dimethylvaleronitrile) (VAZO 52), 2,2′-azobis(isobutyronitrile) (VAZO 64 or AIBN), 2,2′-azobis-2-methylbutyronitrile (VAZO 67), 1,1-azobis(1-cyclohexanecarbonitrile) (VAZO 88); 2,2′-azobis(2-cyclopropylpropionitrile), 2,2′-azobis(methylisobutyrate), 4,4′-Azobis(4-cyanovaleric acid), and combinations thereof.

In accordance with the invention, any photoinitiators can be used in the invention so long as it can generate free radicals for initiating polymerization reaction upon being irradiated with a visible light having a wavelength greater 440 nm. Examples of preferred photoinitiators include without limitation benzoylphosphine photoinitiators, acyl germanium photoinitiators (i.e., germanium-based Type I photoinitiators as described in U.S. Pat. No. 7,605,190), acyltin photoinitiators (e.g., tetrakis(2,4,6-trimethylbenzoyl)stannane or others described in U.S. patent application Ser. No. 18/308,210).

Examples of preferred benzoylphosphine initiators include without limitation 2,4,6-trimethylbenzoyldiphenylphosphine oxide (TPO); 2,4,6-trimethylbenzoylethoxy-phenylphosphine oxide (TPO-L); bis(2,4,6-trimethylbenzoyl)phenylphosphine oxide (BAPO); bis-(2,6-dichlorobenzoyl)-4-N-propylphenyl-phosphine oxide; bis-(2,6-dichlorobenzoyl)-4-N-butylphenylphosphine oxide; lithium phenyl(2,4,6-trimethylbenzoyl) phosphinate (LiTPO).

Examples of preferred acyl germanium photoinitiators include without limitation Bis(4-methoxybenzoyl)diethylgermanium (BMBDE-Ge), dibenzoyldiethylgermanium (DBDE-Ge), tetrakis(2-ethylbenzoyl)-germanium (TEB-Ge).

Examples of preferred polymerizable blue dyes include without limitation 1,4-bis(4-(2-methacryloxyethyl)phenylamino) anthraquinone (Reactive Blue 246), 1,4-bis((2-methacryloxy-ethyl)amino)anthraquinone (Reactive Blue 247).

Nearly any hydrophilic vinylic monomer can be used in the invention. Suitable hydrophilic vinylic monomers have been used in producing non-silicone hydrogel contact lenses and silicone hydrogel contact lenses. Examples of such hydrophilic vinylic monomers include, without limitation, N,N-dimethyl (meth)acrylamide, 2-acrylamidoglycolic acid, N-hydroxypropyl (meth)acrylamide, N-hydroxyethyl (meth)acrylamide, N-[tris(hydroxymethyl)-methyl] (meth)acrylamide, N-vinylpyrrolidone (NVP), N-vinyl formamide, N-vinyl acetamide, N-vinyl isopropylamide, N-vinyl-N-methyl acetamide (VMA), N-methyl-3-methylene-2-pyrrolidone, 1-methyl-5-methylene-2-pyrrolidone, 5-methyl-3-methylene-2-pyrrolidone, 2-hydroxyethyl (meth)acrylate, hydroxypropyl (meth)acrylate, methoxyethyl (meth)acrylate, trimethylammonium 2-hydroxy propylmethacrylate hydrochloride, aminopropyl methacrylate hydrochloride, dimethylaminoethyl methacrylate (DMAEMA), glycerol methacrylate (GMA), a C₁-C₄-alkoxy polyethylene glycol (meth)acrylate having a weight average molecular weight of up to 1500, polyethylene glycol (meth)acrylate having a weight average molecular weight of up to 1500, methacrylic acid, acrylic acid, methacryloxyethyl phosphocholine, methacryloxypropyl phosphocholine, N-2-hydroxyethyl vinyl carbamate, N-carboxyvinyl-β-alanine (VINAL), N-carboxyvinyl-α-alanine, and mixtures thereof.

Any siloxane-containing vinylic monomer can be used in the invention. Examples of preferred siloxane-containing vinylic monomers can be siloxane-containing (meth)acrylamido monomers, siloxane-containing (meth)acryloxy monomers, siloxane-containing vinyloxy-carbonyloxy monomers, siloxane-containing vinyloxycarbonylamino monomers, siloxane-containing vinylaminocarbonylamino monomers, or siloxane-containing vinylaminocarbonyloxy monomers, each of which comprises a bis(trialkylsilyloxy)alkylsilyl group, a tris(trialkylsilyloxy)-silyl group, or a polysiloxane chain having 2 to 30 siloxane units and terminated with an alkyl, hydroxyalkyl or methoxyalkyl group. Such preferred siloxane-containing vinylic monomers can be obtained from the commercial suppliers, or alternatively prepared according to known procedures, e.g., similar to those described in U.S. Pat. Nos. 5,070,215, 6,166,236, 6,867,245, 7,214,809, 8,415,405, 8,475,529, 8,614,261, 8,658,748, 9,097,840, 9,103,965, 9,217,813, 9,315,669, and 9,475,827, or by reacting a vinylic monomer having a reactive functional group (e.g., an acid chloride, acid anhydride, carboxyl, hydroxyl, amino, epoxy, isocyanate, aziridine, azlactone, or aldehyde group) with a siloxane-containing compound having a reactive group selected from the group consisting of a hydroxyalkyl, an aminoalkyl, an alkylaminoalkyl, a carboxyalkyl, an isocyanatoalkyl, an epoxyalkyl, and an aziridinylalkyl, in the presence or absence of a coupling agent under coupling reaction conditions well known to a person skilled in the art.

In accordance with the invention, any polysiloxane vinylic crosslinkers can be used in this invention. Examples of preferred polysiloxane vinylic crosslinkers include without limitation α,ω-(meth)acryloxy-terminated polydimethylsiloxanes of various molecular weight; α,ω-(meth)acrylamido-terminated polydimethylsiloxanes of various molecular weight; α,ω-vinyl carbonate-terminated polydimethylsiloxanes of various molecular weight; α,ω-vinyl carbamate-terminated polydimethylsiloxane of various molecular weight; bis-3-methacryloxy-2-hydroxypropyloxypropyl polydimethylsiloxane of various molecular weight; N,N,N′,N′-tetrakis(3-methacryloxy-2-hydroxypropyl)-alpha,omega-bis-3-aminopropyl-polydimethylsiloxane of various molecular weight; the reaction products of glycidyl methacrylate with amino-functional polydimethylsiloxanes; the reaction products of an azlactone-containing vinylic monomer (any one of those described above) with hydroxyl-functional polydimethylsiloxanes; polysiloxane-containing macromer selected from the group consisting of Macromer A, Macromer B, Macromer C, and Macromer D described in U.S. Pat. No. 5,760,100; polysiloxane vinylic crosslinkers disclosed in U.S. Pat. Nos. 4,136,250, 4,153,641, 4,182,822, 4,189,546, 4,259,467, 4,260,725, 4,261,875, 4,343,927, 4,254,248, 4,355,147, 4,276,402, 4,327,203, 4,341,889, 4,486,577, 4,543,398, 4,605,712, 4,661,575, 4,684,538, 4,703,097, 4,833,218, 4,837,289, 4,954,586, 4,954,587, 5,010,141, 5,034,461, 5,070,170, 5,079,319, 5,039,761, 5,346,946, 5,358,995, 5,387,632, 5,416,132, 5,449,729, 5,451,617, 5,486,579, 5,962,548, 5,981,675, 6,039,913, 6,762,264, 7,423,074, 8,163,206, 8,480,227, 8,529,057, 8,835,525, 8,993,651, 9,187,601, 10,081,697, 10,301,451, and 10,465,047.

One class of preferred polysiloxane vinylic crosslinkers are di-(meth)acryloyloxy-terminated polysiloxane vinylic crosslinkers each having dimethylsiloxane units and hydrophilized siloxane units each having one methyl substituent and one monovalent C₄-C₄₀ organic radical substituent having 2 to 6 hydroxyl groups, more preferably a polysiloxane vinylic crosslinker of formula (H), are described later in this application and can be prepared according to the procedures disclosed in U.S. Pat. No. 10,081,697.

Another class of preferred polysiloxane vinylic crosslinkers are vinylic crosslinkers each of which comprises one sole polysiloxane segment and two terminal (meth)acryloyl groups, which can be obtained from commercial suppliers; prepared by reacting glycidyl (meth)acrylate (meth)acryloyl chloride with a di-amino-terminated polydimethylsiloxane or a di-hydroxyl-terminated polydimethylsiloxane; prepared by reacting isocyantoethyl (meth)acrylate with di-hydroxyl-terminated polydimethylsiloxanes prepared by reacting an amino-containing acrylic monomer with di-carboxyl-terminated polydimethylsiloxane in the presence of a coupling agent (a carbodiimide); prepared by reacting a carboxyl-containing acrylic monomer with di-amino-terminated polydimethylsiloxane in the presence of a coupling agent (a carbodiimide); or prepared by reacting a hydroxyl-containing acrylic monomer with a dihydroxy-terminated polydisiloxane in the presence of a diisocyanate or diepoxy coupling agent.

Other classes of preferred polysiloxane vinylic crosslinkers are chain-extended polysiloxane vinylic crosslinkers each of which has at least two polysiloxane segments linked by a linker between each pair of polysiloxane segments and two terminal ethylenically unsaturated groups, which can be prepared according to the procedures described in U.S. Pat. Nos. 5,034,461, 5,416,132, 5,449,729, 5,760,100, 7,423,074, 8,529,057, 8,835,525, 8,993,651, 9,187,601, 10,301,451, and 10,465,047.

Examples of preferred hydrophobic non-silicone vinylic monomers include without limitation C₁-C₁₀ alkyl (meth)acrylate (e.g., methyl (meth)acrylate, ethyl (meth)acrylate, propyl (meth)acrylate, isopropyl (meth)acrylate, 2-ethylhexyl (meth)acrylate, etc.), cyclohexyl (meth)acrylate, vinyl acetate, vinyl propionate, vinyl butyrate, vinyl valerate, styrene, chloroprene, vinyl chloride, vinylidene chloride, (meth)acrylonitrile, 1-butene, butadiene, vinyl toluene, vinyl ethyl ether, perfluorohexylethyl-thio-carbonyl-aminoethyl-methacrylate, isobornyl (meth)acrylate, trifluoroethyl (meth)acrylate, hexafluoro-isopropyl (meth)acrylate, hexafluorobutyl (meth)acrylate, and combinations thereof.

Examples of preferred non-silicone vinylic crosslinkers include without limitation ethyleneglycol di-(meth)acrylate, diethyleneglycol di-(meth)acrylate, triethyleneglycol di-(meth)acrylate, tetraethyleneglycol di-(meth)acrylate, glycerol di-(meth)acrylate, 1,3-propanediol di-(meth)acrylate, 1,3-butanediol di-(meth)acrylate, 1,4-butanediol di-(meth)acrylate, glycerol 1,3-diglycerolate di-(meth)acrylate, ethylene-bis[oxy(2-hydroxypropane-1,3-diyl)] di-(meth)acrylate, bis[2-(meth)acryloxyethyl] phosphate, trimethylolpropane di-(meth)acrylate, and 3,4-bis[(meth)acryloyl]-tetrahydrofuan, diacrylamide, dimethacrylamide, N,N-di(meth)acryloyl-N-methylamine, N,N-di(meth)acryloyl-N-ethylamine, N,N′-methylene bis(meth)acrylamide, N,N′-ethylene bis(meth)acrylamide, N,N′-dihydroxyethylene bis(meth)acrylamide, N,N′-propylene bis(meth)acrylamide, N,N′-2-hydroxypropylene bis(meth)acrylamide, N,N′-2,3-dihydroxybutylene bis(meth)acrylamide, 1,3-bis(meth)acrylamidepropane-2-yl dihydrogen phosphate, piperazine diacrylamide, tetraethyleneglycol divinyl ether, triethyleneglycol divinyl ether, diethyleneglycol divinyl ether, ethyleneglycol divinyl ether, triallyl isocyanurate, triallyl cyanurate, trimethylopropane trimethacrylate, pentaerythritol tetramethacrylate, bisphenol A dimethacrylate, allylmethacrylate, allylacrylate, N-allyl-methacrylamide, N-allyl-acrylamide, or combinations thereof.

In accordance with the invention, a polymerizable composition of the invention is a fluid composition, which can be a solution (i.e., one including non-reactive diluent—solvent), a solventless blend (i.e., a fluid composition free of any non-reactive diluent—solvent).

Where a polymerizable composition of the invention is a solution. It can be prepared by dissolving all of the desirable components in any suitable solvent known to a person skilled in the art. Example of suitable solvents includes without limitation, water, tetrahydrofuran, tripropylene glycol methyl ether, dipropylene glycol methyl ether, ethylene glycol n-butyl ether, ketones (e.g., acetone, methyl ethyl ketone, etc.), diethylene glycol n-butyl ether, diethylene glycol methyl ether, ethylene glycol phenyl ether, propylene glycol methyl ether, propylene glycol methyl ether acetate, dipropylene glycol methyl ether acetate, propylene glycol n-propyl ether, dipropylene glycol n-propyl ether, tripropylene glycol n-butyl ether, propylene glycol n-butyl ether, dipropylene glycol n-butyl ether, tripropylene glycol n-butyl ether, propylene glycol phenyl ether dipropylene glycol dimetyl ether, polyethylene glycols, polypropylene glycols, ethyl acetate, butyl acetate, amyl acetate, methyl lactate, ethyl lactate, i-propyl lactate, methylene chloride, 2-butanol, 1-propanol, 2-propanol, menthol, cyclohexanol, cyclopentanol and exonorborneol, 2-pentanol, 3-pentanol, 2-hexanol, 3-hexanol, 3-methyl-2-butanol, 2-heptanol, 2-octanol, 2-nonanol, 2-decanol, 3-octanol, norborneol, tert-butanol, tert-amyl, alcohol, 2-methyl-2-pentanol, 2,3-dimethyl-2-butanol, 3-methyl-3-pentanol, 1-methylcyclohexanol, 2-methyl-2-hexanol, 3,7-dimethyl-3-octanol, 1-chloro-2-methyl-2-propanol, 2-methyl-2-heptanol, 2-methyl-2-octanol, 2-2-methyl-2-nonanol, 2-methyl-2-decanol, 3-methyl-3-hexanol, 3-methyl-3-heptanol, 4-methyl-4-heptanol, 3-methyl-3-octanol, 4-methyl-4-octanol, 3-methyl-3-nonanol, 4-methyl-4-nonanol, 3-methyl-3-octanol, 3-ethyl-3-hexanol, 3-methyl-3-heptanol, 4-ethyl-4-heptanol, 4-propyl-4-heptanol, 4-isopropyl-4-heptanol, 2,4-dimethyl-2-pentanol, 1-methylcyclopentanol, 1-ethylcyclopentanol, 1-ethylcyclopentanol, 3-hydroxy-3-methyl-1-butene, 4-hydroxy-4-methyl-1-cyclopentanol, 2-phenyl-2-propanol, 2-methoxy-2-methyl-2-propanol 2,3,4-trimethyl-3-pentanol, 3,7-dimethyl-3-octanol, 2-phenyl-2-butanol, 2-methyl-1-phenyl-2-propanol and 3-ethyl-3-pentanol, 1-ethoxy-2-propanol, 1-methyl-2-propanol, t-amyl alcohol, isopropanol, 1-methyl-2-pyrrolidone, N,N-dimethylpropionamide, dimethyl formamide, dimethyl acetamide, dimethyl propionamide, N-methyl pyrrolidinone, and mixtures thereof. Preferably, a polymerizable composition is a solution of all the desirable components in water, 1,2-propylene glycol, a polyethyleneglycol having a molecular weight of about 400 Daltons or less, or a mixture thereof.

Where a polymerizable composition of the invention is a solventless blend, it can be prepared by mixing all polymerizable components and other necessary component. A solventless polymerizable composition typically comprises at least one blending vinylic monomer as a reactive solvent for dissolving all other polymerizable components of the solventless polymerizable composition. Examples of preferred blending vinylic monomers are described later in this application. Preferably, methyl methacrylate is used as a blending vinylic monomer in preparing a solventless polymerizable composition.

Numerous lens formulations (polymerizable compositions) for making non-silicone hydrogel contact lenses have been described in numerous patents and patent applications published by the filing date of this application and have been used in producing commercial non-silicone hydrogel contact lenses. Examples of commercial non-silicone hydrogel contact lenses include, without limitation, alfafilcon A, acofilcon A, deltafilcon A, etafilcon A, focofilcon A, helfilcon A, helfilcon B, hilafilcon B, hioxifilcon A, hioxifilcon B, hioxifilcon D, methafilcon A, methafilcon B, nelfilcon A, nesofilcon A, ocufilcon A, ocufilcon B, ocufilcon C, ocufilcon D, omafilcon A, phemfilcon A, polymacon, samfilcon A, telfilcon A, tetrafilcon A, and vifilcon A. They can be used as a base formulation for forming a polymerizable composition for forming a HEVL-filtering non-silicone hydrogel contact lens of the invention by adding at least one UV-absorbing vinylic monomer (any one described above), at least one HEVL-absorbing vinylic monomer of the invention (described above), and at least one polymerizable blue dye (described above) or Cu(II) phthalocyanine blue pigment particles in the base formulation.

Numerous lens formulations (polymerizable compositions) for forming silicone hydrogel contact lenses have been described in numerous patents and patent applications published by the filing date of this application and have been used in producing commercial SiHy contact lenses. Examples of commercial SiHy contact lenses include, without limitation, asmofilcon A, balafilcon A, comfilcon A, delefilcon A, efrofilcon A, enfilcon A, fanfilcon A, galyfilcon A, lotrafilcon A, lotrafilcon B, narafilcon A, narafilcon B, senofilcon A, senofilcon B, senofilcon C, smafilcon A, somofilcon A, and stenfilcon A. They can be used as a base formulation for forming a polymerizable composition for forming a HEVL-filtering silicone hydrogel contact lens of the invention by adding at least one UV-absorbing vinylic monomer (any one described above), at least one HEVL-absorbing vinylic monomer of the invention (described above), and at least one polymerizable blue dye (described above) or Cu(II) phthalocyanine blue pigment particles in the base formulation.

Lens molds for making hydrogel contact lenses are well known to a person skilled in the art and, for example, are employed in cast molding or spin casting. For example, a mold (for cast molding) generally comprises at least two mold sections (or portions) or mold halves, i.e., first and second mold halves. The first mold half defines a first molding (or optical) surface and the second mold half defines a second molding (or optical) surface. The first and second mold halves are configured to receive each other such that a lens-forming cavity is formed between the first molding surface and the second molding surface. The molding surface of a mold half is the cavity-forming surface of the mold and in direct contact with the polymerizable composition.

The mold halves can be formed through various techniques, such as injection molding. Methods of manufacturing mold halves for cast-molding a contact lens are generally well known to those of ordinary skill in the art. The process of the present invention is not limited to any particular method of forming a mold. In fact, any method of forming a mold can be used in the present invention. The mold halves can be formed through various techniques, such as injection molding or lathing. Examples of suitable processes for forming the mold halves are disclosed in U.S. Pat. Nos. 4,444,711; 4,460,534; 5,843,446; and 5,894,002.

Virtually all materials known in the art for making molds can be used to make molds for making contact lenses. For example, polymeric materials, such as polyethylene, polypropylene, polystyrene, PMMA, Topas® COC grade 8007-S10 (clear amorphous copolymer of ethylene and norbornene, from Ticona GmbH of Frankfurt, Germany and Summit, New Jersey), or the like can be used. Other materials that allow UV light transmission could be used, such as quartz glass and sapphire.

In accordance with the invention, a polymerizable composition can be introduced (dispensed) into a cavity formed by a mold according to any known methods.

After the polymerizable composition is dispensed into the mold, it is polymerized (cured) to produce a contact lens. Curing (polymerizing) may be initiated thermally or actinically to polymerize (cure) the polymerizable components in the polymerizable composition.

The thermal polymerization is carried out conveniently, for example at a temperature of from 25 to 120° C. and preferably 40 to 100° C. The reaction time may vary within wide limits, but is conveniently, for example, from 30 minutes to 4 hours or preferably from 1 to 2 hours. It is advantageous to previously degas the components and solvents used in the polymerization reaction and to carry out said copolymerization reaction under an inert atmosphere, for example under a nitrogen or argon atmosphere.

The actinic polymerization can then be triggered off by actinic radiation, for example, a visible light of a suitable wavelength (e.g., ≥450 nm).

After the curing step, the steps of opening a mold (i.e., separating the male mold half from the female mold half with the hydrogel contact lens attached onto one of the male and female mold halves and delensing (i.e., removing the hydrogel contact lens from the lens adhered mold half) are carried out according to any techniques known to a skilled person.

After the molded hydrogel contact lens is delensed, it typically is extracted with an extraction medium as well known to a person skilled in the art. The extraction liquid medium is any solvent capable of dissolving the diluent(s), unpolymerized polymerizable materials, and oligomers in the lens precursor. Water, any organic solvents described above or known to a person skilled in the art, or a mixture thereof can be used in the invention.

The extracted hydrogel contact lens can then be hydrated according to any method known to a person skilled in the art.

The extracted and/or hydrated hydrogel contact lens can further subject to further processes, such as, for example, surface treatment, packaging in lens packages with a packaging solution which is well known to a person skilled in the art; sterilization such as autoclave at from 118 to 124° C. for at least about 30 minutes; and the like.

Lens packages (or containers) are well known to a person skilled in the art for autoclaving and storing a soft contact lens. Any lens packages can be used in the invention. Preferably, a lens package is a blister package which comprises a base and a cover, wherein the cover is detachably sealed to the base, wherein the base includes a cavity for receiving a sterile packaging solution and the contact lens.

Hydrogel contact lenses are packaged in individual packages, sealed, and sterilized (e.g., autoclave at 120° C. or higher for at least 30 minutes under pressure) prior to dispensing to users. A person skilled in the art will understand well how to seal and sterilize lens packages.

In a still further aspect, the invention provides a HEVL-filtering hydrogel contact lens comprising a crosslinked polymeric material which comprises: (1) repeating units of (a) at least one UV-absorbing vinylic monomer, (b) at least one HEVL-absorbing benzotriazole vinylic monomer of formula (I) or (II) (as defined above), and (c) at least one polymerizable components selected from the group consisting of a hydrophilic vinylic monomer, a siloxane-containing vinylic monomer, a polysiloxane vinylic crosslinker, a non-silicone vinylic crosslinker, a non-silicone hydrophobic vinylic monomer, and combinations thereof; and (2) Cu(II)-phthalocyanine blue pigment particles distributed therein and/or repeating units of a polymerizable blue dye, wherein the HEVL-filtering hydrogel contact lens has a UVA % T of less than 5%, UVB%T of about 1% or less, a HEVL % T of about 35% or less (preferably about 32% or less, more preferably about 30% or less, even more preferably about 28% or less), and a color expressed with a*≤−5 (preferably a*=−20 to −6) and b*≤+18.

Various embodiments of UV-absorbing vinylic monomers, hydrophilic vinylic monomers, siloxane-containing vinylic monomers, polysiloxane vinylic crosslinkers, non-silicone hydrophobic vinylic monomers, non-silicone vinylic crosslinkers, and polymerizable blue dyes have been described above and can be used in this aspect of the invention.

The CIE (Commission Internationale de L′Eclairage) has standardized color order systems. The CIE L*a*b* Color System is used in the invention to express a color by the numbers. The lightness value, L*, also referred to as “Lstar,” defines black at 0 and white at 100. The at axis is relative to the green-magenta opponent colors, with negative values toward green and positive values toward magenta. The b* axis represents the blue-yellow opponents, with negative numbers toward blue and positive toward yellow. Those values can be determined in color measurements by X-rite, as illustrated in examples.

A HEVL-filtering hydrogel contact lens of the invention has at least one property selected from the group consisting of: an oxygen permeability of at least about 60 barrers (preferably at least about 70 barrers, more preferably at least about 80 barrers) (at about 35° C.); an elastic modulus of about 1.5 MPa or less (preferably about 1.2 MPa or less, more preferably from about 0.3 MPa to about 1.0 MPa) (at a temperature of from about 22° C. to 28° C.); an equilibrium water content of from about 15% to about 75% (preferably from about 20% to about 70%, more preferably from about 25% to about 65%) by weight (at room temperature) when being fully hydrated.

Although various embodiments of the invention have been described using specific terms, devices, and methods, such description is for illustrative purposes only. The words used are words of description rather than of limitation. It is to be understood that changes and variations may be made by those skilled in the art without departing from the spirit or scope of the present invention, which is set forth in the following claims. In addition, it should be understood that aspects of the various embodiments may be interchanged either in whole or in part or can be combined in any manner and/or used together, as illustrated below:

1. A HEVL-absorbing benzotriazole vinylic monomer of any one of formula (I) or (II)

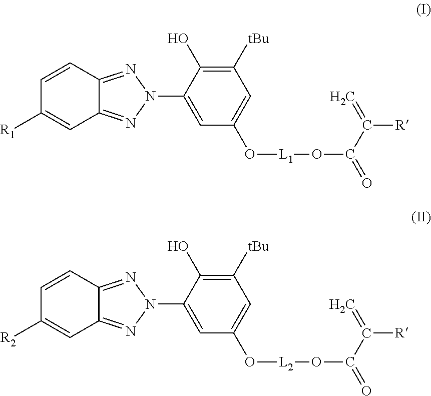

in which: tBu is tert-butyl group, R′ is H or CH₃, R₁ is Cl or CF₃, L₁ is a divalent C₅-C₆ alkylene group or a divalent group of —C₂H₄—(OC₂H₄)ₙ—, R₂ is an organic group which is free of any halogen atom and has a Hammett substituent constant (σₚ) of from about 0.15to about 0.70 (preferably from about 0.20 to about 0.65), L₂ is a divalent C₂-C₁₀ alkylene group or a divalent group of —C₂H₄—(OC₂H₄)ₙ—, n is 1, 2 or 3.
   2. The HEVL-absorbing benzotriazole vinylic monomer of embodiment 1, being of formula (I).
   3. The HEVL-absorbing benzotriazole vinylic monomer of embodiment 1, being of formula (II).
   4. The HEVL-absorbing benzotriazole vinylic monomer of embodiment 3, wherein R₂ has a Hammett substituent constant (σₚ) of from about 0.20 to about 0.65.
   5. The HEVL-absorbing benzotriazole vinylic monomer of embodiment 3 or 4, wherein R₂ is —CH₂CN, —OCOCH₃, —CO(CH₃)₃, —SO₃, —CONH₂, —OSO₂CH₃, —CONHCH₃, —CON(CH₃)₂, —CH₂N⁺(CH₃)₃, —COOH, —COOCH₃, —COOC₂H₅, —COC₂H₅, —COCH₃, or —CN.
   6. A method for making HEVL-filtering hydrogel contact lenses, comprising the steps of:
(1) obtaining a polymerizable composition comprising
   (a) from about 0.1% to about 2.5% by weight of (preferably from about 0.2% to about 2.0% by weight of, more preferably from about 0.3% to about 1.8% by weight of, even more preferably from about 0.4% to about 1.6% by weight of) at least one UV-absorbing vinylic monomer,
   (b) from about 0.2% to about 3.0% by weight of (preferably from about 0.25% to about 2.5% by weight of, more preferably from about 0.3% to about 2.5% by weight of, even more preferably from about 0.35% to about 2.25% by weight of) at least one HEVL-absorbing benzotriazole vinylic monomer of any one of embodiments 1 to 5,
   (c) from about 0.1% to about 2.0% by weight of (preferably from about 0.25% to about 1.75% by weight of, more preferably from about 0.5% to about 1.5% by weight of, even more preferably from about 0.75% to about 1.25% by weight of) at least free-radical initiator,
   (d) at least one blue-tinting agent which is Cu(II)-phthalocyanine blue pigment particles or at least one polymerizable blue dye, and
   (e) at least one polymerizable component selected from the group consisting of a hydrophilic vinylic monomer, a siloxane-containing vinylic monomer, a polysiloxane vinylic crosslinker, a non-silicone vinylic crosslinker, a non-silicone hydrophobic vinylic monomer, and combinations thereof;
(2) introducing the polymerizable composition into a mold for making a hydrogel contact lens, wherein the mold has a first mold half with a first molding surface defining the anterior surface of the contact lens and a second mold half with a second molding surface defining the posterior surface of the contact lens, wherein said first and second mold halves are configured to receive each other such that a cavity is formed between said first and second molding surfaces; and
(3) curing thermally or actinically the lens formulation in the mold to form the HEVL-filtering hydrogel contact lens, wherein the HEVL-filtering hydrogel contact lens has a UVA % T of less than 5%, UVB % T of about 1% or less, a HEVL % T of about 35% or less (preferably about 32% or less, more preferably about 30% or less, even more preferably about 28% or less).
   7. The method of embodiment 6, wherein the free-radical initiator is a thermal initiator, wherein the step of curing is carried out thermally.
   8. The method of embodiment 6, wherein the free-radical initiator is a photoinitiator, wherein the step of curing is carried out by irradiation with a visible light having a wavelength within the range from 450 nm to 510 nm.
   9. The method of embodiment 8, wherein the photoinitiator is a benzoylphosphine oxide photoinitiator, an acyl germanium photoinitiator, an acyltin photoinitiator, or combinations thereof.
   10. The method of embodiment 8, wherein the photoinitiator is an acyl germanium photoinitiator.
   11. A HEVL-filtering hydrogel contact lens, comprising a crosslinked polymeric material which comprises:
(1) repeating units of
   (a) at least one UV-absorbing vinylic monomer,
   (b) at least one HEVL-absorbing benzotriazole vinylic monomer of any one of embodiments 1 to 5, and
   (c) at least one polymerizable component selected from the group consisting of a hydrophilic vinylic monomer, a siloxane-containing vinylic monomer, a polysiloxane vinylic crosslinker, a non-silicone vinylic crosslinker, a non-silicone hydrophobic vinylic monomer, and combinations thereof; and
(2) Cu(II)-phthalocyanine blue pigment particles distributed therein and/or repeating units of a polymerizable blue dye, wherein the HEVL-filtering hydrogel contact lens has a UVA % T of less than 5%, UVB%T of about 1% or less, a HEVL % T of about 35% or less, and a color expressed with a*≤−5 and b*≤+18.
   12. The HEVL-filtering hydrogel contact lens of embodiment 11, wherein the UV/HEVL-filtering hydrogel contact lens has a HEVL % T of about 32% or less.
   13. The HEVL-filtering hydrogel contact lens of embodiment 11, wherein the UV/HEVL-filtering hydrogel contact lens has a HEVL % T of about 30% or less.
   14. The HEVL-filtering hydrogel contact lens of embodiment 11, wherein the UV/HEVL-filtering hydrogel contact lens has a HEVL % T of about 28% or less.
   15. The HEVL-filtering hydrogel contact lens of any one of embodiments 11 to 14, wherein the UV/HEVL-filtering hydrogel contact lens has a color expressed with a*=−20 to −6 and b*≤+18.
   16. The HEVL-filtering hydrogel contact lens of any one of embodiments 11 to 15, wherein the crosslinked polymeric material comprises at least one polymerizable blue dye which is 1,4-bis((2-methoxyethyl)amino)-anthraquinone and/or 1,4-bis(4-(2-methacryloxyethyl)phenylamino)-anthraquinone.
   17. The HEVL-filtering hydrogel contact lens of any one of embodiments 11 to 15, wherein the crosslinked polymeric material comprises Cu(II)-phthalocyanine blue pigment particles distributed therein.
   18. The HEVL-filtering hydrogel contact lens of any one of embodiments 11 to 17, wherein said at least one UV-absorbing vinylic monomer comprises a benzotriazole-containing vinylic monomer.
   19. The UV/HEVL-filtering hydrogel contact lens of embodiment 18, wherein said at least one UV-absorbing vinylic monomer comprises 2-(2′-hydroxy-5′-vinylphenyl)-2H-benzotriazole, 2-(2′-hydroxy-5′-methacryloxyphenyl)-2H-benzotriazole, 2-(2′-hydroxy-5′-acryloyloxyphenyl)-2H-benzotriazole, 2-[2′-hydroxy-5′-(2-methacryloxyethyl)phenyl)]-2H-benzotriazole (Norbloc), 2-[2′-hydroxy-5′-(2-acryloxyethyl) phenyl)]-2H-benzotriazole, 2-(2′-hydroxy-5′-methacryloxypropyl-phenyl)-2H-benzotriazole, 2-(2′-hydroxy-5′-acryloxypropylphenyl)-2H-benzotriazole, or combinations thereof.
   20. The HEVL-filtering hydrogel contact lens of embodiment 18, wherein said at least one UV-absorbing vinylic monomer is 2-[2′-hydroxy-5′-(2-methacryloxyethyl)phenyl)]-2H-benzotriazole.
   21. The HEVL-filtering hydrogel contact lens of any one of embodiments 11 to 20, wherein said at least one HEVL-absorbing benzotriazole vinylic monomer comprises a HEVL-absorbing benzotriazole vinylic monomer of embodiment 2.
   22. The HEVL-filtering hydrogel contact lens of any one of embodiments 11 to 21, wherein said at least one HEVL-absorbing benzotriazole vinylic monomer comprises a HEVL-absorbing benzotriazole vinylic monomer of embodiments 3 to 5.
   23. The HEVL-filtering hydrogel contact lens of any one of embodiments 11 to 22, wherein the crosslinked polymeric material comprises repeating units of at least one hydrophilic vinylic monomer selected from the group consisting of N,N-dimethyl (meth)acrylamide, 2-acrylamidoglycolic acid, N-hydroxypropyl (meth)acrylamide, N-hydroxyethyl (meth)acrylamide, N-[tris (hydroxymethyl)-methyl] (meth)acrylamide, N-vinylpyrrolidone, N-vinyl formamide, N-vinyl acetamide, N-vinyl isopropylamide, N-vinyl-N-methyl acetamide, N-methyl-3-methylene-2-pyrrolidone, 1-methyl-5-methylene-2-pyrrolidone, 5-methyl-3-methylene-2-pyrrolidone, 2-hydroxyethyl (meth)acrylate, hydroxypropyl (meth)acrylate, methoxyethyl (meth)acrylate, trimethylammonium 2-hydroxy propylmethacrylate hydrochloride, aminopropyl methacrylate hydrochloride, dimethylaminoethyl methacrylate (DMAEMA), glycerol methacrylate (GMA), a C₁-C₄-alkoxy polyethylene glycol (meth)acrylate having a weight average molecular weight of up to 1500, polyethylene glycol (meth)acrylate having a weight average molecular weight of up to 1500, methacrylic acid, acrylic acid, methacryloxyethyl phosphocholine, methacryloxypropyl phosphocholine, N-2-hydroxyethyl vinyl carbamate, N-carboxyvinyl-β-alanine, N-carboxyvinyl-α-alanine, and mixtures thereof.
   24. The HEVL-filtering hydrogel contact lens of any one of embodiments 11 to 23, wherein the crosslinked polymeric material is a silicone hydrogel material that comprises repeating units of at least one siloxane-containing vinylic monomer and/or at least one polysiloxane vinylic crosslinker.
   25. The HEVL-filtering hydrogel contact lens of embodiment 24, wherein the silicone hydrogel material comprises repeating units of said at least one siloxane-containing vinylic monomer selected from the group consisting of α-(meth)acryloxypropyl terminated ω-C₁-C₄-alkyl terminated polydimethylsiloxane, α-(meth)acryloxy-2-hydroxypropyloxypropyl terminated ω-C₁-C₄-alkyl terminated polydimethylsiloxane, α-(2-hydroxyl-methacryloxy-propyloxypropyl)-ω-[3-C₁-C₄-alkyl-decamethylpentasiloxane, α-[3-(meth)acryloxyethoxy-2-hydroxypropyloxypropyl]-terminated ω-C₁-C₄-alkyl terminated polydimethylsiloxane, α-[3-(meth)acryloxy-propyloxy-2-hydroxypropyloxypropyl]-terminated ω-C₁-C₄-alkyl terminated polydimethylsiloxane, α-[3-(meth)acryloxyisopropyloxy-2-hydroxypropyloxypropyl]-terminated ω-C₁-C₄-alkyl terminated polydimethylsiloxane, α-[3-(meth)acryloxybutyloxy-2-hydroxypropyloxypropyl]-terminated ω-C₁-C₄-alkyl terminated polydimethylsiloxane, α-[3-(meth)acryloxyethylamino-2-hydroxypropyloxypropyl]-terminated ω-C₁-C₄-alkyl terminated polydimethylsiloxane, α-[3-(meth)acryloxypropylamino-2-hydroxypropyloxypropyl]-terminated ω-C₁-C₄-alkyl terminated polydimethylsiloxane, α-[3-(meth)acryloxy-butylamino-2-hydroxypropyloxypropyl]-terminated ω-C₁-C₄-alkyl terminated polydimethylsiloxane, α-(meth)acryloxy(polyethylenoxy)-2-hydroxypropyloxypropyl]-terminated ω-C₁-C₄-alkyl terminated polydimethylsiloxane, α-[(meth)acryloxy-2-hydroxypropyloxy-ethoxypropyl]-terminated ω-C₁-C₄-alkyl terminated polydimethylsiloxane, α-[(meth)acryloxy-2-hydroxypropyl-N-ethylaminopropyl]-terminated ω-C₁-C₄-alkyl terminated polydimethylsiloxane, α-[(meth)acryloxy-2-hydroxypropylamino-propyl]-terminated ω-C₁-C₄-alkyl terminated polydimethylsiloxane, α-[(meth)acryloxy-2-hydroxypropyloxy-(polyethylenoxy)propyl]-terminated ω-C₁-C₄-alkyl terminated polydimethylsiloxane, α-(meth)acryloylamidopropyloxypropyl terminated ω-C₁-C₄-alkyl terminated polydimethylsiloxane, α-N-methyl-(meth)acryloylamidopropyloxypropyl terminated ω-C₁-C₄-alkyl terminated polydimethylsiloxane, α-[3-(meth)acrylamidoethoxy-2-hydroxypropyloxy-propyl]-terminated ω-C₁-C₄-alkyl polydimethylsiloxane, α-[3-(meth)acrylamidopropyloxy-2-hydroxypropyloxypropyl]-terminated ω-C₁-C₄-alkyl terminated polydimethylsiloxane, α-[3-(meth)acrylamidoisopropyloxy-2-hydroxypropyloxy-propyl]-terminated ω-C₁-C₄-alkyl terminated polydimethylsiloxane, α-[3-(meth)acrylamido-butyloxy-2-hydroxypropyloxypropyl]-terminated ω-C₁-C₄-alkyl terminated polydimethylsiloxane, α-[3-(meth)acryloylamido-2-hydroxypropyloxypropyl] terminated ω-C₁-C₄-alkyl polydimethylsiloxane, α-[3-[N-methyl-(meth)acryloylamido]-2-hydroxy-propyloxypropyl] terminated ω-C₁-C₄-alkyl terminated polydimethylsiloxane, N-methyl-N′-(propyltetra(dimethylsiloxy)dimethylbutylsilane) (meth)acrylamide, N-(2,3-dihydroxy-propane)-N′-(propyltetra(dimethylsiloxy)dimethylbutylsilane) (meth)acrylamide, (meth)acryloylamidopropyltetra(dimethylsiloxy)dimethylbutylsilane, α-vinyl carbonate-terminated ω-C₁-C₄-alkyl-terminated polydimethylsiloxanes, α-vinyl carbamate-terminated ω-C₁-C₄-alkyl-terminated polydimethylsiloxane, and a mixture thereof.
   26. The HEVL-filtering hydrogel contact lens of embodiment 24, the silicone hydrogel material comprises repeating units of said at least one siloxane-containing vinylic monomer selected from the group consisting of a vinylic monomer having a bis(trialkylsilyloxy)-alkylsilyl group, a vinylic monomer having a tris(trialkylsilyloxy)silyl group, a polysiloxane vinylic monomer, 3-methacryloxy propylpentamethyldisiloxane, t-butyldimethyl-siloxyethyl vinyl carbonate, trimethylsilylethyl vinyl carbonate, and trimethylsilylmethyl vinyl carbonate, and combinations thereof.
   27. The HEVL-filtering hydrogel contact lens of embodiment 24, the silicone hydrogel material comprises repeating units of said at least one siloxane-containing vinylic monomer selected from the group consisting of tris(trimethylsilyloxy)silylpropyl (meth)acrylate, [3-(meth)acryloxy-2-hydroxypropyloxy]propylbis(trimethylsiloxy)-methylsilane, [3-(meth)acryloxy-2-hydroxypropyloxy]propylbis(trimethylsiloxy)butylsilane, 3-(meth)acryloxy-2-(2-hydroxyethoxy)-propyloxy)propylbis(trimethylsiloxy)methylsilane, 3-(meth)acryloxy-2-hydroxypropyloxy) propyltris(trimethylsiloxy)silane, N-[tris(trimethylsiloxy)-silylpropyl]-(meth)acrylamide, N-(2-hydroxy-3-(3-(bis(trimethylsilyloxy)methylsilyl)-propyloxy)propyl)-2-methyl (meth)acrylamide, N-(2-hydroxy-3-(3-(bis(trimethylsilyloxy)-methylsilyl)propyloxy)-propyl) (meth)acrylamide, N-(2-hydroxy-3-(3-(tris(trimethylsilyloxy)-silyl)propyloxy)propyl)-2-methyl acrylamide, N-(2-hydroxy-3-(3-(tris(trimethylsilyloxy)silyl)-propyloxy)propyl) (meth)acrylamide, N-[tris(dimethylpropylsiloxy)silylpropyl]-(meth)acrylamide, N-[tris(dimethylphenylsiloxy)-silylpropyl] (meth)acrylamide, N-[tris(dimethylethylsiloxy)-silylpropyl] (meth)acrylamide, N,N-bis[2-hydroxy-3-(3-(bis(trimethylsilyloxy)methylsilyl)-propyloxy)propyl]-2-methyl (meth)acrylamide, N,N-bis[2-hydroxy-3-(3-(bis(trimethyl-silyloxy)methylsilyl)propyloxy)-propyl] (meth)acrylamide, N,N-bis[2-hydroxy-3-(3-(tris(trimethylsilyloxy)silyl)propyloxy)-propyl]-2-methyl (meth)acrylamide, N,N-bis[2-hydroxy-3-(3-(tris(trimethylsilyloxy)silyl)-propyloxy)propyl] (meth)acrylamide, N-[2-hydroxy-3-(3-(t-butyldimethylsilyl)propyloxy)-propyl]-2-methyl (meth)acrylamide, N-[2-hydroxy-3-(3-(t-butyldimethylsilyl)propyloxy)-propyl] (meth)acrylamide, N,N-bis[2-hydroxy-3-(3-(t-butyldimethylsilyl)propyloxy)propyl]-2-methyl (meth)acrylamide, N-2-(meth)acryloxyethyl-O-(methyl-bis-trimethylsiloxy-3-propyl)silyl carbamate, 3-(trimethylsilyl)propylvinyl carbonate, 3-(vinyloxycarbonylthio)-propyl-tris(trimethyl-siloxy)silane, 3-[tris(trimethyl-siloxy)silyl]propylvinyl carbamate, 3-[tris(trimethylsiloxy)silyl] propyl allyl carbamate, 3-[tris(trimethylsiloxy)silyl] propyl vinyl carbonate, and combinations thereof.
   28. The HEVL-filtering hydrogel contact lens of any one of embodiments 11 to 27, wherein the silicone hydrogel material comprises repeating units of said at least one polysiloxane vinylic crosslinker.
   29. The HEVL-filtering contact lens of embodiment 28, wherein said at least one polysiloxane vinylic crosslinker comprises a di-(meth)acryloyloxy-terminated polysiloxane vinylic crosslinker having dimethylsiloxane units and hydrophilized siloxane units each having one methyl substituent and one monovalent C₄-C₄₀ organic radical substituent having 2 to 6 hydroxyl groups.
   30. The HEVL-filtering hydrogel contact lens of embodiment 28, wherein said at least one polysiloxane vinylic crosslinker comprises at least one hydrophilized polysiloxane vinylic crosslinker of formula (G)

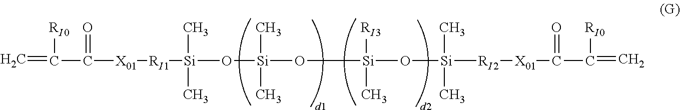

in which:
d1 is an integer of from 30 to 500 and d2 is an integer of from 1 to 75, provided that
d2/d1 is from about 0.035 to about 0.15;
X₀₁ is O or NR_{IN} in which R_{IN} is hydrogen or C₁-C₁₀-alkyl;
R_{I0} is hydrogen or methyl;
R_{I1} and R_{I2} independently of each other are a substituted or unsubstituted C₁-C₁₀ alkylene divalent radical or a divalent radical of-R_{I4}-O-R_{I5}-in which R_{I4} and R_{I5}independently of each other are a substituted or unsubstituted C₁-C₁₀ alkylene divalent radical;
R_{I3} is a monovalent radical of any one of formula (G-1) to (G-5)

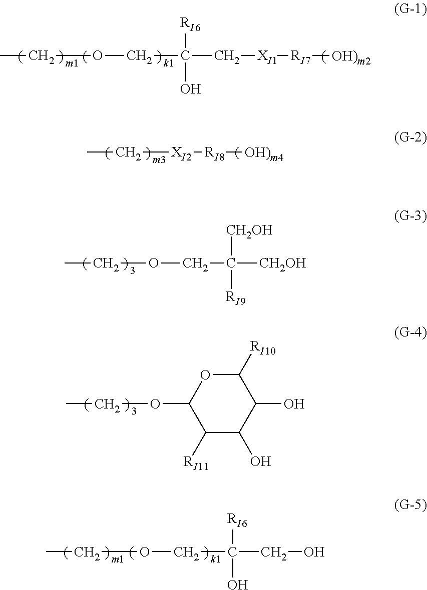

k1 is zero or 1; m1 is an integer of 2 to 4; m2 is an integer of 1 to 5; m3 is an integer of 3 to 6; m4 is an integer of 2 to 5;
R_{I6} is hydrogen or methyl;
R_{I7} is a C₂-C₆ hydrocarbon radical having (m2+1) valencies;
R_{I8} is a C₂-C₆ hydrocarbon radical having (m4+1) valencies;
R_{I9} is ethyl or hydroxymethyl;
R_{I10} is methyl or hydromethyl;
R_{I11} is hydroxyl or methoxy;
X_{I1} is a sulfur linkage of —S — or a teriary amino linkage of —NR_{I12}— in which R_{I12} is C₁-C₁ alkyl, hydroxyethyl, hydroxypropyl, or 2,3-dihydroxypropyl; and

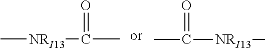

X_{I2} is an amide linkage of in which R_{I13} is hydrogen or C₁-C₁₀ alkyl.
   31. The HEVL-filtering hydrogel contact lens of embodiment 28, wherein said at least one polysiloxane vinylic crosslinker comprises: (i) a vinylic crosslinker which comprises one sole polydiorganosiloxane segment and two terminal ethylenically-unsaturated groups selected from the group consisting of (meth)acryloyloxy groups, (meth)acryloylamino groups, vinyl carbonate groups, vinylcarbamate groups; and/or (ii) a chain-extended polysiloxane vinylic crosslinker which comprises at least two polydiorganosiloxane segment and a covalent linker between each pair of polydiorganosiloxane segments and two two terminal ethylenically-unsaturated groups selected from the group consisting of (meth)acryloyloxy groups, (meth)acryloylamino groups, vinyl carbonate groups, vinylcarbamate groups.
   32. The HEVL-filtering hydrogel contact lens of embodiment 28, wherein said at least one polysiloxane vinylic crosslinker comprises α,ω-bis[3-(meth)acrylamidopropyl]-terminated polydimethylsiloxane, α,ω-bis[3-(meth)acryloxypropyl]-terminated polydimethylsiloxane, α,ω-bis[3-(meth)acryloxy-2-hydroxypropyloxypropyl]-terminated polydimethylsiloxane, α,ω-bis[3-(meth)acryloxyethoxy-2-hydroxypropyloxypropyl]-terminated polydimethylsiloxane, α,ω-bis[3-(meth)acryloxypropyloxy-2-hydroxypropyloxypropyl]-terminated polydimethylsiloxane, α,ω-bis[3-(meth)acryloxy-isopropyloxy-2-hydroxypropyloxypropyl]-terminated polydimethylsiloxane, α,ω-bis[3-(meth)acryloxybutyloxy-2-hydroxypropyloxypropyl]-terminated polydimethylsiloxane, α,ω-bis[3-(meth)acrylamidoethoxy-2-hydroxypropyloxypropyl]-terminated polydimethylsiloxane, α,ω-bis[3-(meth)acrylamidopropyloxy-2-hydroxypropyloxypropyl]-terminated polydimethylsiloxane, α,ω-bis[3-(meth)acrylamidoisopropyloxy-2-hydroxypropyloxypropyl]-terminated polydimethylsiloxane, α,ω-bis[3-(meth) acrylamidobutyloxy-2-hydroxypropyloxypropyl]-terminated polydimethylsiloxane, α,ω-bis[3-(meth)acryloxyethylamino-2-hydroxypropyloxypropyl]-terminated polydimethylsiloxane, α,ω-bis[3-(meth)acryloxypropylamino-2-hydroxypropyloxypropyl]-terminated polydimethylsiloxane, α,ω-bis[3-(meth)acryloxybutylamino-2-hydroxypropyloxypropyl]-terminated polydimethylsiloxane, α,ω-bis[(meth)acrylamidoethylamino-2-hydroxypropyloxy-propyl]-terminated polydimethylsiloxane, α,ω-bis[3-(meth)acrylamidopropylamino-2-hydroxypropyloxypropyl]-terminated polydimethylsiloxane, α,ω-bis[3-(meth)acrylamide-butylamino-2-hydroxypropyloxypropyl]-terminated polydimethylsiloxane, α,ω-bis[(meth)acryloxy-2-hydroxypropyloxy-ethoxypropyl]-terminated polydimethylsiloxane, α,ω-bis[(meth)acryloxy-2-hydroxypropyl-N-ethylaminopropyl]-terminated polydimethylsiloxane, α,ω-bis[(meth)acryloxy-2-hydroxypropyl-aminopropyl]-polydimethylsiloxane, α,ω-bis[(meth)acryloxy-2-hydroxypropyloxy-(polyethylenoxy)propyl]-terminated polydimethylsiloxane, α,ω-bis[(meth)acryloxyethylamino-carbonyloxy-ethoxypropyl]-terminated polydimethylsiloxane, α,ω-bis[(meth)acryloxyethylamino-carbonyloxy-(polyethylenoxy)propyl]-terminated polydimethylsiloxane, or combinations thereof.

33. The HEVL-filtering hydrogel contact lens of any one of embodiments 24 to 32, wherein said at least one hydrophilic vinylic monomer comprises at least one hydrophilic N-vinyl amide monomer selected from the group consisting of N-vinylpyrrolidone, N-vinyl piperidone, N-vinyl caprolactam, N-vinyl-N-methyl acetamide, N-vinyl formamide, N-vinyl acetamide, N-vinyl isopropylamide, N-vinyl-N-methyl acetamide, N-vinyl-N-ethyl acetamide, N-vinyl-N-ethyl formamide, and mixtures thereof.

34. The HEVL-filtering hydrogel contact lens of any one of embodiments 24 to 32, wherein said at least one hydrophilic vinylic monomer comprises N-vinylpyrrolidone and/or N-vinyl-N-methyl acetamide.
35. The HEVL-filtering hydrogel contact lens of any one of embodiments 24 to 32, wherein said at least one hydrophilic vinylic monomer comprises (meth)acrylamide, N,N-dimethyl (meth)acrylamide, N-ethyl (meth)acrylamide, N,N-diethyl (meth)acrylamide, N-propyl (meth)acrylamide, N-isopropyl (meth)acrylamide, N-3-methoxypropyl (meth)acrylamide, N-2-hydroxylethyl (meth)acrylamide, N,N-bis(hydroxyethyl) (meth)acrylamide, N-3-hydroxypropyl (meth)acrylamide, N-2-hydroxypropyl (meth)acrylamide, N-2,3-dihydroxypropyl (meth)acrylamide, N-tris(hydroxymethyl)methyl (meth)acrylamide, poly(ethylene glycol)ethyl (meth)acrylamide having a number average molecular weight of up to 1500, 2-(meth)acrylamidoglycolic acid, 3-(meth)acrylamidopropionic acid, 5-(meth)acrylamidopentanoic acid, 4-(meth)acrylamidobutanoic acid, 3-(meth)acrylamido-2-methylbutanoic acid, 3-(meth)acrylamido-3-methylbutanoic acid, 2-(meth)acrylamido-2-methyl-3,3-dimethyl butanoic acid, 3-(meth)acrylamidohaxanoic acid, 4-(meth)acrylamido-3,3-dimethylhexanoic acid, N-2-aminoethyl (meth)acrylamide, N-2-methylaminoethyl (meth)acrylamide, N-2-ethylaminoethyl (meth)acrylamide, N-2-dimethylaminoethyl (meth)acrylamide, N-3-aminopropyl (meth)acrylamide, N-3-methylaminopropyl (meth)acrylamide, N-3-dimethylaminopropyl (meth)acrylamide, methoxy-poly(ethylene glycol)ethyl (meth)acrylamide having a number average molecular weight of up to 1500, or combinations thereof.
36. The HEVL-filtering hydrogel contact lens of any one of embodiments 24 to 32, wherein said at least one hydrophilic vinylic monomer comprises (meth)acrylamide, N,N-dimethyl (meth)acrylamide, N-ethyl (meth)acrylamide, N-propyl (meth)acrylamide, N-isopropyl (meth)acrylamide, N-3-methoxypropyl (meth)acrylamide, N-2-hydroxylethyl (meth)acrylamide, N-3-hydroxypropyl (meth)acrylamide, or combinations thereof.
37. The HEVL-filtering hydrogel contact lens of any one of embodiments 24 to 36, wherein said at least one hydrophilic vinylic monomer comprises hydroxyethyl (meth) acrylate, glycerol (meth)acrylate, 3-hydroxypropyl (meth)acrylate, 2-hydroxypropyl (meth)acrylate, 3-amino-2-hydroxypropyl (meth)acrylate, N-2-hydroxyethyl (meth)acrylamide, N-3-hydroxypropyl (meth)acrylamide, N-2-hydroxypropyl (meth)acrylamide, N-2,3-dihydroxypropyl (meth)acrylamide, N-tris(hydroxymethyl)methyl (meth)acrylamide, vinyl alcohol, allyl alcohol, or combinations thereof.
38. The HEVL-filtering hydrogel contact lens of any one of embodiments 24 to 37, wherein said at least one hydrophilic vinylic monomer comprises 1-methyl-3-methylene-2-pyrrolidone, 1-ethyl-3-methylene-2-pyrrolidone, 1-methyl-5-methylene-2-pyrrolidone, 1-ethyl-5-methylene-2-pyrrolidone, 5-methyl-3-methylene-2-pyrrolidone, 5-ethyl-3-methylene-2-pyrrolidone, 1-n-propyl-3-methylene-2-pyrrolidone, 1-n-propyl-5-methylene-2-pyrrolidone, 1-isopropyl-3-methylene-2-pyrrolidone, 1-isopropyl-5-methylene-2-pyrrolidone, 1-n-butyl-3-methylene-2-pyrrolidone, 1-tert-butyl-3-methylene-2-pyrrolidone, or combinations thereof.
39. The HEVL-filtering hydrogel contact lens of any one of embodiments 24 to 38, wherein the HEVL-filtering hydrogel contact lens has an oxygen permeability of at least 60 barrers (at about 35° C.) when being fully hydrated.
40. The HEVL-filtering hydrogel contact lens of any one of embodiments 24 to 38, wherein the HEVL-filtering hydrogel contact lens has an oxygen permeability of at least 70 barrers (at about 35° C.) when being fully hydrated.
41. The HEVL-filtering hydrogel contact lens of any one of embodiments 24 to 38, wherein the HEVL-filtering hydrogel contact lens has an oxygen permeability of at least 80 barrers (at about 35° C.) when being fully hydrated.
42. The HEVL-filtering hydrogel contact lens of any one of embodiments 24 to 41, wherein the HEVL-filtering hydrogel contact lens has an elastic modulus of about 2.0 MPa or less (at a temperature of from 22° C. to 28° C.) when being fully hydrated.
43. The HEVL-filtering hydrogel contact lens of any one of embodiments 24 to 41, wherein the HEVL-filtering hydrogel contact lens has an elastic modulus of about 1.5 MPa or less (at a temperature of from 22° C. to 28° C.) when being fully hydrated.
44. The HEVL-filtering hydrogel contact lens of any one of embodiments 24 to 41, wherein the HEVL-filtering hydrogel contact lens has an elastic modulus of about 1.2 or less (at a temperature of from 22° C. to 28° C.) when being fully hydrated.
45. The HEVL-filtering hydrogel contact lens of any one of embodiments 24 to 41, wherein the HEVL-filtering hydrogel contact lens has an elastic modulus of from about 0.4 MPa to about 1.0 MPa (at a temperature of from 22° C. to 28° C.) when being fully hydrated.
46. The HEVL-filtering hydrogel contact lens of any one of embodiments 24 to 45, wherein the HEVL-filtering hydrogel contact lens has a water content of from about 15% to about 70% (at a temperature of from 22° C. to 28° C.) when being fully hydrated.
47. The HEVL-filtering hydrogel contact lens of any one of embodiments 24 to 45, wherein the HEVL-filtering hydrogel contact lens has a water content of from about 20% to about 70% (at a temperature of from 22° C. to 28° C.) when being fully hydrated.
48. The HEVL-filtering hydrogel contact lens of any one of embodiments 24 to 45, wherein the HEVL-filtering hydrogel contact lens has a water content of from about 25% to about 70% (at a temperature of from 22° C. to 28° C.) when being fully hydrated.
49. The HEVL-filtering hydrogel contact lens of any one of embodiments 24 to 45, wherein the HEVL-filtering hydrogel contact lens has a water content of from about 30% to about 65% (at a temperature of from 22°° C. to 28° C.) when being fully hydrated.
50. The HEVL-filtering hydrogel contact lens of any one of embodiments 24 to 49, wherein the silicone hydrogel material further comprises repeating units of at least one non-silicone vinylic crosslinker.
51. The HEVL-filtering hydrogel contact lens of embodiment 50, wherein said at least one non-silicone vinylic crosslinker comprises ethyleneglycol di-(meth)acrylate, diethyleneglycol di-(meth)acrylate, triethyleneglycol di-(meth)acrylate, tetraethyleneglycol di-(meth)acrylate, glycerol di-(meth)acrylate, 1,3-propanediol di-(meth)acrylate, 1,3-butanediol di-(meth)acrylate, 1,4-butanediol di-(meth)acrylate, glycerol 1,3-diglycerolate di-(meth)acrylate, ethylenebis[oxy(2-hydroxypropane-1,3-diyl)] di-(meth)acrylate, bis[2-(meth)acryloxyethyl] phosphate, trimethylolpropane di-(meth)acrylate, and 3,4-bis[(meth)acryloyl]tetrahydrofuan, diacrylamide, dimethacrylamide, N,N-di(meth)acryloyl-N-methylamine, N,N-di(meth)acryloyl-N-ethylamine, N,N′-methylene bis(meth)acrylamide, N,N′-ethylene bis(meth)acrylamide, N,N′-dihydroxyethylene bis(meth)acrylamide, N,N′-propylene bis(meth)acrylamide, N,N′-2-hydroxypropylene bis(meth)acrylamide, N,N′-2,3-dihydroxybutylene bis(meth)acrylamide, 1,3-bis(meth)acrylamidepropane-2-yl dihydrogen phosphate, piperazine diacrylamide, tetraethyleneglycol divinyl ether, triethyleneglycol divinyl ether, diethyleneglycol divinyl ether, ethyleneglycol divinyl ether, triallyl isocyanurate, triallyl cyanurate, trimethylopropane trimethacrylate, pentaerythritol tetramethacrylate, bisphenol A dimethacrylate, allylmethacrylate, allylacrylate, N-allyl-methacrylamide, N-allyl-acrylamide, or combinations thereof.
52. The HEVL-filtering hydrogel contact lens of any one of embodiments 24 to 51, wherein the silicone hydrogel material further comprises repeating units of at least one hydrophobic non-silicone vinylic monomer.
53. The HEVL-filtering hydrogel contact lens of embodiment 52, wherein said at least one hydrophobic non-silicone vinylic monomer comprises C₁-C₁₀ alkyl (meth)acrylate, cyclopentylacrylate, cyclohexylmethacrylate, cyclohexylacrylate, isobornyl (meth)acrylate, styrene, 4,6-trimethylstyrene (TMS), t-butyl styrene (TBS), trifluoroethyl (meth)acrylate, hexafluoro-isopropyl (meth)acrylate, hexafluorobutyl (meth)acrylate, or combinations thereof.
54. The HEVL-filtering hydrogel contact lens of embodiment 30, wherein the silicone hydrogel material comprises: repeating units of said at least one polysiloxane vinylic monomer recited in embodiment 30; repeating units of at least one hydrophilic N-vinyl amide monomer selected from the group consisting of N-vinylpyrrolidone, N-vinyl piperidone, N-vinyl caprolactam, N-vinyl-N-methyl acetamide, N-vinyl formamide, N-vinyl acetamide, N-vinyl isopropylamide, N-vinyl-N-methyl acetamide, N-vinyl-N-ethyl acetamide, N-vinyl-N-ethyl formamide, and mixtures thereof; repeating units of at least one non-silicone vinylic crosslinker; repeating units of at least one hydrophobic non-silicone vinylic monomer selected from the group consisting of C₁-C₁₀ alkyl (meth)acrylate, cyclopentylacrylate, cyclohexylmethacrylate, cyclohexylacrylate, isobornyl (meth)acrylate, styrene, 4,6-trimethylstyrene (TMS), t-butyl styrene (TBS), trifluoroethyl (meth)acrylate, hexafluoro-isopropyl (meth) acrylate, hexafluorobutyl (meth)acrylate, and combinations thereof.
55. The HEVL-filtering hydrogel contact lens of embodiment 54, wherein said at least one hydrophilic N-vinyl amide monomer is N-vinylpyrrolidone and/or N-vinyl-N-methyl acetamide, wherein said at least one hydrophobic non-silicone vinylic monomer is methyl methacrylate, wherein said at least one non-silicone vinylic crosslinker comprises ethyleneglycol di-(meth)acrylate, diethyleneglycol di-(meth)acrylate, triethyleneglycol di-(meth)acrylate, tetraethyleneglycol di-(meth)acrylate, glycerol di-(meth)acrylate, triallyl isocyanurate, allyl methacrylate, or combinations thereof.
56. The HEVL-filtering hydrogel contact lens of embodiment 24, wherein the silicone hydrogel material in dried state comprises at least 90% by weight of repeating units of (a) at least hydrophilic (meth)acrylamido monomer as said at least one hydrophilic vinylic monomer; (b) at least one siloxane-containing (meth)acrylamido monomer as said at least one siloxane-containing vinylic monomer; and (c) said at least one polysiloxane vinylic monomer.
57. The HEVL-filtering hydrogel contact lens of embodiment 56, wherein said at least one hydrophilic (meth) acrylamido monomer comprises (meth)acrylamide, N,N-dimethyl (meth)acrylamide, N-ethyl (meth)acrylamide, N,N-diethyl (meth)acrylamide, N-propyl (meth)acrylamide, N-isopropyl (meth)acrylamide, N-3-methoxypropyl (meth)acrylamide, N-2-hydroxylethyl (meth)acrylamide, N,N-bis(hydroxyethyl) (meth)acrylamide, N-3-hydroxypropyl (meth)acrylamide, N-2-hydroxypropyl (meth)acrylamide, N-2,3-dihydroxypropyl (meth)acrylamide, N-4-hydroxybutyl (meth)acrylamide, N,N-bis(2-hydroxyethyl) (meth)acrylamide, N-tris(hydroxymethyl) methyl (meth)acrylamide, 2-(meth)acrylamido-glycolic acid, 3-(meth)acrylamido-propionic acid, 4-(meth)acrylamido-butanoic acid, 3-(meth)acrylamido-2-methylbutanoic acid, 3-(meth)acrylamido-3-methylbutanoic acid, 2-(meth)acrylamido-2-methyl-3,3-dimethyl butanoic acid, 5-(meth)acrylamidopentanoic acid, 3-(meth)acrylamidohaxanoic acid, 4-(meth)acrylamido-3,3-dimethylhexanoic acid, (3-(meth)acrylamidophenyl)boronic acid, 3- ((3-methacrylamidopropyl)dimethylammonio)-propane-1-sulfonate; 3-((3-acrylamidopropyl)dimethylammonio)propane-1-sulfonate, N-2-aminoethyl (meth)acrylamide, N-2-methylaminoethyl (meth)acrylamide, N-2-ethylamino-ethyl (meth)acrylamide, N-2-dimethylaminoethyl (meth)acrylamide, N-3-aminopropyl (meth)acrylamide, N-3-methylaminopropyl (meth)acrylamide, N-3-dimethylaminopropyl (meth)acrylamide, poly (ethylene glycol) ethyl (meth)acrylamide having a number average molecular weight of up to 700, methoxy-poly(ethylene glycol)ethyl (meth)acrylamide having a number average molecular weight of up to 700, or combination thereof.
58. The HEVL-filtering hydrogel contact lens of embodiment 56, wherein said at least one hydrophilic (meth)acrylamido monomer comprises N,N-dimethyl (meth)acrylamide, N-ethyl (meth)acrylamide, N-2-hydroxyethyl (meth)acrylamide, N-3-hydroxypropyl (meth)acrylamide, N-2-hydroxypropyl (meth)acrylamide, N-2,3-dihydroxypropyl (meth)acrylamide, (meth) acrylamide, N-(2-aminoethyl) (meth)acrylamide, N-(3-aminopropyl) (meth)acrylamide, or combinations thereof.
59. The HEVL-filtering hydrogel contact lens of embodiment 56, wherein said at least one hydrophilic (meth) acrylamido monomer comprises N,N-dimethyl (meth)acrylamide.
60. The HEVL-filtering hydrogel contact lens of any one of embodiments 56 to 59, wherein said at least one siloxane-containing (meth)acrylamido monomer comprises a (meth)acrylamido monomer containing a tris(trialkylsiloxy)silyl group.
61. The HEVL-filtering hydrogel contact lens of embodiment 60, wherein the (meth)acrylamido monomer containing a tris(trialkylsiloxy)silyl group is N-[tris(trimethylsiloxy)silylpropyl] (meth)acrylamide, N-[tris(dimethylethylsiloxy)-silylpropyl] (meth)acrylamide, N-[tris(dimethylpropylsiloxy)silylpropyl]acrylamide, N-[tris(dimethylphenylsiloxy)silylpropyl] (meth)acrylamide, N-(2-hydroxy-3-(3-(tris(trimethylsilyloxy)silyl)propyloxy)propyl)-2-methyl acrylamide, N-(2-hydroxy-3-(3-(tris(trimethylsilyloxy)silyl)propyloxy)propyl)acrylamide, N,N-bis[2-hydroxy-3-(3-(tris(trimethylsilyloxy)silyl)propyloxy)propyl]-2-methyl acrylamide, N,N-bis[2-hydroxy-3-(3-(tris(trimethylsilyloxy)silyl)propyloxy)propyl] acrylamide, or a combination thereof.
62. The HEVL-filtering hydrogel contact lens of any one of embodiments 56 to 59, wherein said at least one siloxane-containing (meth)acrylamido monomer comprises a (meth) acrylamido monomer containing a bis(trialkylsilyloxy)alkylsilyl group.
63. The HEVL-filtering hydrogel contact lens of any one of embodiments 56 to 59, wherein said at least one siloxane-containing (meth)acrylamido monomer comprises a (meth)acrylamido monomer containing a polysiloxane segment having 2 to 20 siloxane units.
64. The HEVL-filtering hydrogel contact lens of any one of embodiments 56 to 63, wherein said at least one polysiloxane vinylic crosslinker comprises: an α,ω-(meth)acryloxy-terminated polydimethylsiloxane; an α,ω-(meth)acrylamido-terminated polydimethylsiloxane; an α,ω-vinyl carbonate-terminated polydimethylsiloxane; an α,ω-vinyl carbamate-terminated polydimethylsiloxane; a bis-3-methacryloxy-2-hydroxypropyloxypropyl polydimethylsiloxane; N,N,N′,N′-tetrakis(3-methacryloxy-2-hydroxypropyl)-alpha, omega-bis-3-aminopropyl-polydimethylsiloxane of various molecular weight; a reaction product of glycidyl methacrylate with a di-amino-functionalized polydimethylsiloxane; a reaction product of an azlactone-containing vinylic monomer with a d-hydroxyl-functionalized polydimethylsiloxane; or combinations thereof.
65. The HEVL-filtering hydrogel contact lens of any one of embodiments 56 to 63, wherein said at least one polysiloxane vinylic crosslinker comprises: (1) a vinylic crosslinker which comprises one sole polydiorganosiloxane segment and two terminal ethylenically-unsaturated groups selected from the group consisting of (meth)acryloyloxy groups, (meth)acryloylamino groups, vinyl carbonate groups, vinylcarbamate groups; and/or (2) a chain-extended polysiloxane vinylic crosslinker which comprises at least two polydiorganosiloxane segment and a covalent linker between each pair of polydiorganosiloxane segments and two two terminal ethylenically-unsaturated groups selected from the group consisting of (meth)acryloyloxy groups, (meth)acryloylamino groups, vinyl carbonate groups, vinylcarbamate groups.
66. The HEVL-filtering hydrogel contact lens of any one of embodiments 56 to 63, wherein said at least one polysiloxane vinylic crosslinker comprises α,ω-bis[3-(meth)acrylamidopropyl]-terminated polydimethylsiloxane, α,ω-bis[3-(meth)acryloxypropyl]-terminated polydimethylsiloxane, α,ω-bis[3-(meth)acryloxy-2-hydroxypropyloxypropyl]-terminated polydimethylsiloxane, α,ω-bis[3-(meth)acryloxyethoxy-2-hydroxypropyloxypropyl]-terminated polydimethylsiloxane, α,ω-bis[3-(meth)acryloxypropyloxy-2-hydroxypropyloxy-propyl]-terminated polydimethylsiloxane, α,ω-bis[3-(meth)acryloxy-isopropyloxy-2-hydroxypropyloxypropyl]-terminated polydimethylsiloxane, α,ω-bis[3-(meth)acryloxy-butyloxy-2-hydroxypropyloxypropyl]-terminated polydimethylsiloxane, α,ω-bis[3-(meth)acrylamidoethoxy-2-hydroxypropyloxypropyl]-terminated polydimethylsiloxane, α,ω-bis[3-(meth)acrylamidopropyloxy-2-hydroxypropyloxypropyl]-terminated polydimethylsiloxane, α,ω-bis[3-(meth)acrylamidoisopropyloxy-2-hydroxypropyloxypropyl]-terminated polydimethylsiloxane, α,ω-bis[3-(meth)acrylamidobutyloxy-2-hydroxy-propyloxypropyl]-terminated polydimethylsiloxane, α,ω-bis[3-(meth)acryloxyethylamino-2-hydroxypropyloxypropyl]-terminated polydimethylsiloxane, α,ω-bis[3-(meth)acryloxypropyl-amino-2-hydroxypropyloxypropyl]-terminated polydimethylsiloxane, α,ω-bis[3-(meth)acryloxybutylamino-2-hydroxypropyloxypropyl]-terminated polydimethylsiloxane, α,ω-bis[(meth)acrylamidoethylamino-2-hydroxypropyloxy-propyl]-terminated polydimethylsiloxane, α,ω-bis[3-(meth)acrylamidopropylamino-2-hydroxypropyloxypropyl]-terminated polydimethylsiloxane, α,ω-bis[3-(meth)acrylamide-butylamino-2-hydroxypropyloxypropyl]-terminated polydimethylsiloxane, α,ω-bis[(meth)acryloxy-2-hydroxypropyloxy-ethoxypropyl]-terminated polydimethylsiloxane, α,ω-bis[(meth)acryloxy-2-hydroxypropyl-N-ethylaminopropyl]-terminated polydimethylsiloxane, α,ω-bis[(meth)acryloxy-2-hydroxypropyl-aminopropyl]-polydimethylsiloxane, α,ω-bis[(meth)acryloxy-2-hydroxypropyloxy-(polyethylenoxy)propyl]-terminated polydimethylsiloxane, α,ω-bis[(meth)acryloxyethylamino-carbonyloxy-ethoxypropyl]-terminated polydimethylsiloxane, α,ω-bis[(meth)acryloxyethylamino-carbonyloxy-(polyethylenoxy)propyl]-terminated polydimethylsiloxane, or combinations thereof.
67. The HEVL-filtering hydrogel contact lens of any one of embodiments 11 to 23, wherein the crosslinked polymeric material is a non-silicone hydrogel material.
68. The HEVL-filtering hydrogel contact lens of embodiment 67, wherein the non-silicone hydrogel material comprises repeating units of a hydroxy-containing vinylic monomer.
69. The HEVL-filtering hydrogel contact lens of embodiment 68, wherein the hydroxy-containing vinylic monomer is selected from the group consisting of hydroxyethyl (meth)acrylate, glycerol (meth)acrylate, 3-hydroxypropyl (meth)acrylate, 2-hydroxypropyl (meth)acrylate, 3-amino-2-hydroxypropyl (meth)acrylate, N-2-hydroxyethyl (meth)acrylamide, N-3-hydroxypropyl (meth)acrylamide, N-2-hydroxypropyl (meth)acrylamide, N-2,3-dihydroxypropyl (meth)acrylamide, N-tris(hydroxymethyl)methyl (meth)acrylamide, vinyl alcohol, allyl alcohol, and combinations thereof.
70. The HEVL-filtering hydrogel contact lens of embodiment 67, wherein the non-silicone hydrogel material comprises repeating units of N-vinylpyrrolidone, N-vinyl-N-methyl acetamide, 1-methyl-3-methylene-2-pyrrolidone, or combinations thereof.

The previous disclosure will enable one having ordinary skill in the art to practice the invention. Various modifications, variations, and combinations can be made to the various embodiment described herein. In order to better enable the reader to understand specific embodiments and the advantages thereof, reference to the following examples is suggested. It is intended that the specification and examples be considered as exemplary.

### EXAMPLE 1

### Oxygen Permeability Measurements

Unless specified, the oxygen transmissibility (Dk/t), the intrinsic (or edge-corrected) oxygen permeability (Dkᵢ or Dk_{c}) of a lens and a lens material are determined according to procedures described in ISO 18369-4.

### Equilibrium Water Content

The equilibrium water content (EWC) of contact lenses are determined as follows. Amount of water (expressed as percent by weight) present in a hydrated hydrogel contact lens, which is fully equilibrated in saline solution, is determined at room temperature. Quickly stack the lenses and transfer the lens stack to the aluminum pan on the analytical balance after blotting lens in a cloth. The number of lenses for each sample pan is typically five (5). Record the pan plus hydrated weight of the lenses. Cover the pan with aluminum foil. Place pans in a laboratory oven at 100±2° C. to dry for 16-18 hours. Remove pan plus lenses from the oven and cool in a desiccator for at least 30 minutes. Remove a single pan from the desiccator, and discard the aluminum foil. Weigh the pan plus dried lens sample on an analytical balance. Repeat for all pans. The wet and dry weight of the lens samples can be calculated by subtracting the weight of the empty weigh pan.

### Elastic Modulus

The elastic modulus of a contact lens is determined using a MTS insight instrument. The contact lens is first cut into a 3.12 mm wide strip using Precision Concept two stage cutter. Five thickness values are measured within 6.5 mm gauge length. The strip is mounted on the instrument grips and submerged in PBS (phosphate buffered saline) with the temperature controlled at 21±2° C. Typically 5N Load cell is used for the test. Constant force and speed is applied to the sample until the sample breaks. Force and displacement data are collected by the TestWorks software. The elastic modulus value is calculated by the TestWorks software which is the slope or tangent of the stress vs. strain curve near zero elongation, in the elastic deformation region.

### Transmittance

Contact lenses are manually placed into a specially fabricated sample holder or the like which can maintain the shape of the lens as it would be when placing onto eye. This holder is then submerged into a 1 cm path-length quartz cell containing phosphate buffered saline (PBS, pH˜7.0-7.4) as the reference. A UV/visible spectrpohotmeter, such as, Varian Cary 3E UV-Visible Spectrophotometer with a LabSphere DRA-CA-302 beam splitter or the like, can be used in this measurement. Percent transmission spectra are collected at a wavelength range of 250-800 nm with % T values collected at 0.5 nm intervals. This data is transposed onto an Excel spreadsheet and used to determine if the lenses conform to Class 1 UV absorbance. Transmittance is calculated using the following equations:

$$UVA \% T = Average \% Transmission between 315 nm and 380 nm \times 100$$

$$UVB \% T = Average \% Transmission between 280 nm and 315 nm \times 100$$

$$HEVL \% T = Average \% Transmission between 380 nm and 450 nm \times 100.$$

### Chemicals

CE-PDMS represents a polysiloxane vinylic crosslinker (Mw=10.1 K determined by ¹H NMR spectroscopy) which has three polydimethylsiloxane (PDMS) segments linked via diurethane linkages between two PDMS segments and two urethane linkages each located between one terminal methacrylate group and one PDMS segment and is prepared according to method similar to what described in Example 2 of U.S. Pat. No. 9315669; TRIS-Am represents N-[tris(trimethylsiloxy)-silylpropyl]acrylamide; mPDMS represents monomethacryloxypropyl-terminated butyl terminated polydimethylsiloxane (Mw: 600-800 Daltons); MMA represents methyl methacrylate; NVP represents N-vinylpyrrolidone; EGMA represents ethylene glycol methyl ether methacrylate; TEGDMA represents triethyleneglycol dimethacrylate; AMA represents allyl methacrylate; DMA represent N,N-dimethylacrylamide; L-PEG 2000 represents N-(carbonyl-methoxypolyethylene glycol-2000)-1,2-disteaoyl-sn-glycero-3-phosphoethanolamin, sodium salt; DMPC represents 1,2-dimyristoyl-sn-glycero-3-phosphocholine; H-TEMPO represents 4-hydroxy-2,2,6,6-tetramethylpiperidin-1-oxyl; Norbloc is 2-[2′-hydroxy-5′-(2-methacryloxyethyl) phenyl)]-2H-benzotriazole from Aldrich; PrOH represents n-propanol; UV28represents 2-{2′-Hydroxy-3′-tert-butyl-5′-[3′-methacryloyloxypropoxy]phenyl}-5-chloro-2H-benzotriazole; UV35 represents 2-propenoic acid, 2-methyl-, 2-[2-[3-(5-chloro-2H-benzotriazol-2-yl)-5-(1,1-dimethylethyl)-4-hydroxyphenoxy]ethoxy]ethyl ester; RB247 represents 1,4-bis((2-methacryloxyethyl)amino)-anthraquinone (Reactive Blue 247); VAZO 64 represents 2,2′-dimethyl-2,2′azodipropiononitrile; BMBDE-Ge represents Bis(4-methoxybenzoyl)diethylgermanium; TEB-Ge represents tetrakis(2-ethylbenzoyl)-germanium; MEK represents methylethyl ketone; PBS represents a phosphate-buffered saline which has a pH of 7.2±0.2 at 25° C. and contains about 0.044 wt. % NaH₂PO₄·H₂O, about 0.388 wt. % Na₂HPO₄·2H₂O, and about 0.79 wt. % NaCl and 98.78% water; wt. % represents weight percent; “G1” represents a di-methacryloyloxypropyl-terminated polysiloxane (Mn˜7.5-8.1 K g/mol, OH content˜1.25-1.55 mmol/g) of formula (A) shown below.

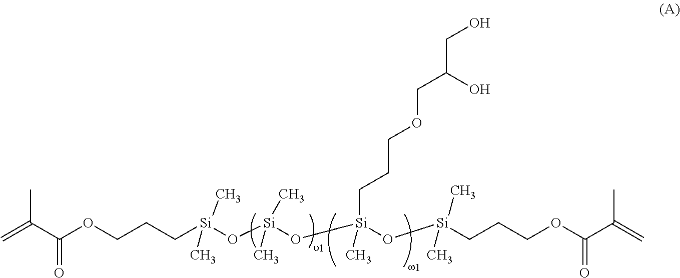

### EXAMPLE 2

This example illustrates the correlation between the melting point and solubility of HEVL-absorbing vinylic monomers of

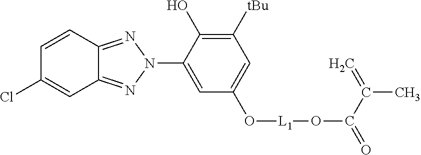

in which L₁ is —(CH₂)₃— or —(CH₂)₂—O—(CH₂)₂—

It is reported that there are correlations between lower melting points and compound solubility (Bathori, N.B. Acta Cryst., 2014, A70, C990).

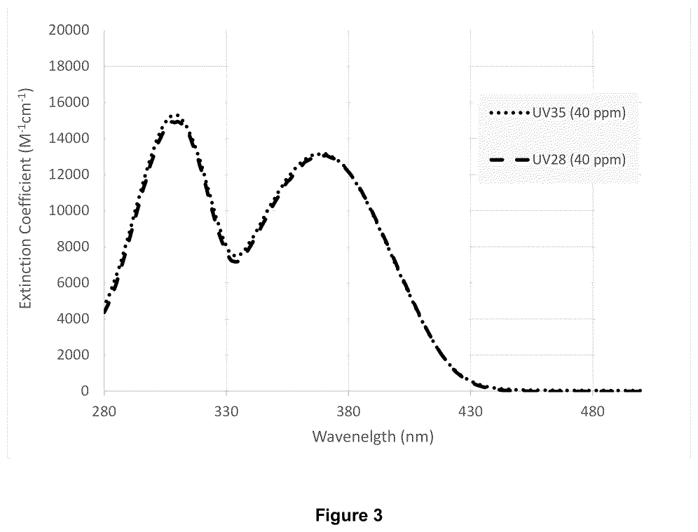

It is found that extending the linker L₁ from —(CH₂)₃— in UV28 to —(CH₂)₂—O—(CH₂)₂— in UV35 lowers the melting point significantly, from 112-113° C. to 62-65° C., respectively. As anticipated, solubility in 1-propanol is improved, reaching >4.0 mg/ml (Table 1). However, the UV/visible absorption profiles of UV28 and UV35 are equivalent, as shown in FIG. 3.

TABLE 1

| | UV28 | UV35 |
|---|---|---|
| R group | —Cl | —Cl |
| L1 Linker | —(CH₂)₃— | —(CH₂)₂—O—(CH₂)₂— |
| Melting point (° C.) | 112-113 | 62-65 |
| Solubility in PrOH (mg/mL) | 1.5 | >4.0 |

### EXAMPLE 3

### Preparation of Lens Formulations

Two lens formulations are prepared to have the compositions shown in Table 2.

TABLE 2

| | Lens Formulation (Wt %) | |
|---|---|---|
| Components | 3-1 | 3-2 |
| H-TEMPO | 0.04 | |
| DMA | 23.12 | |
| 1-PrOH | 23.20 | |
| UV35 | 0 | 1.06 |
| UV28 | 0.20 | 0 |
| Norbloc | 0.60 | 0 |
| DMPC | 0.76 | |
| L-PEG2000 | 0.61 | |
| TRIS-Am | 19.81 | |
| CE-PDMS | 31.67 | |
| TEB-Ge | 0 | 0.75 |
| BMBDE-Ge | 0.60 | 0 |

Photorheology

| | | |
|---|---|---|
| Cure times (s) | 30 | 30 |
| G′ (kPa) | 122 | 116 |

All the components (excluding Germanium based photoinitiators) listed in Table 2 are added into an amber vial. Then under yellow light, TEB-Ge or BMBDE-Ge (the structure of which is listed in Table 3) is added to the vial and all components are mixed for 30 min in the water bath preheated to 40° C. After all the solid is dissolved, the formulation is filtered with a glass micro filter (5.0 μm Millex®-SV filter).

TABLE 3

| Photoinitiator | BMBDE-Ge | TEB-Ge |
|---|---|---|
| Structure | 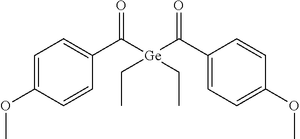 | 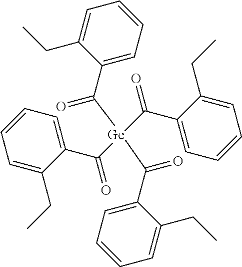 |
| Absorption edge (nm) | 470 | 490 |

PAA-coating solution. A polyacrylic acid (PAA) coating solution is prepared by dissolving an amount of PAA (M.W.: 450 kDa, from Lubrizol) in a given volume of 1-propanol (1-PrOH) to have a concentration of about 0.44% by weight and the pH is adjusted with formic acid to about 2.0. Preparation of In-Package-Coating solution (IPC saline). Poly (AAm-co-AA) (90/10) partial sodium salt (˜90% solid content, poly (AAm-co-AA) 90/10, Mw 200,000) is purchased from Polysciences, Inc. and used as received. Polyamidoamine epichlorohydrin (PAE) (Kymene, an azetidinium content of 0.46 assayed with NMR) is purchased from Ashland as an aqueous solution and used as received. IPC saline is prepared by dissolving about 0.07% w/w of poly (AAm-co-AA) (90/10) and about 0.10% of PAE (an initial azetidinium millimolar equivalents of about 8.8 millimole) in phosphate-buffered saline (PBS) (about 0.044 w/w % NaH₂PO₄·H₂O, about 0.388 w/w/% Na₂HPO₄·2H₂O, about 0.79 w/w % NaCl) and adjusting the pH to 7.2˜7.6. Then the IPC is heat pre-treated for about 6 hours at about 60° C. (heat pretreatment). During this heat pretreatment, poly (AAm-co-AA) and PAE are partially crosslinked to each other (i.e., not consuming all azetidinium groups of PAE) to form a water-soluble and thermally-crosslinkable hydrophilic polymeric material containing azetidinium groups within the branched polymer network in the IPC saline. After the heat pre-treatment, the IPC is cooled to room temperature then filtered using a 0.22 micron PES membrane filter.

### Lens Fabrication (Lightstream)

Lenses are prepared by cast-molding from the lens-forming composition prepared above in a reusable mold (quartz female mold half and glass male mold half), similar to the mold shown in FIGS. 1-6 in U.S. Pat. Nos. 7,384,590 and 7,387,759 (FIGS. 1-6). Lens formulation in the molds is cured for 30 seconds with 452 nm (formulation with BMBDE-Ge) or 464 nm (formulation with TEB-Ge) LED light at a total intensity of 50 mW/cm². After demolding and delensing, cast-molded contact lenses are then extracted and coated by dipping in the following series of baths: 3 methyl ethyl ketone (MEK) baths (about 22, 78, and 224 seconds respectively); DI water bath (56 seconds); PAA dip solution prepared above (44 seconds); 50/50 n-propanol/water bath (56 seconds); 4 DI water baths (56 seconds each), then equilibrated into PBS solution.

The resultant silicone hydrogel contact lenses are packaged/sealed in polypropylene lens packaging shells (or blisters) (one lens per shell) containing 0.65 mL of IPC saline prepared above and autoclaved for 45 minutes at 121° C.

### EXAMPLE 4

### Preparation of Lens Formulations

A lens formulations (4-1) is prepared to have the composition shown in Table 4.

TABLE 4

| Components | Wt % |
|---|---|
| mPDMS | 32.95 |
| G1 | 5.81 |
| N-Vinyl-2-Pyrrolidone (NVP) | 38.76 |
| Methyl Methacrylate (MMA) | 8.73 |
| Ethylene glycol methyl ether methacrylate (EGMA) | 9.88 |
| Trieethylene glycol dimethacrylate (TEGMA) | 0.29 |
| Allyl Methacrylate (AMA) | 0.10 |
| Norbloc | 0.00 |
| VAZO 64 | 0.49 |
| RB247 | 0.01 |
| 2-Methyl-2-Butanol | 0.97 |
| UV35 | 2.00 |

### Lens Fabrication (Thermal Cure)

Lenses are prepared by cast-molding from a polymerizable composition shown in Table 4. The polymerizable composition is introduced into polypropylene molds and thermally cured in an oven under the following curing profile: ramp from room temperature to 55° C. at a ramp rate of about 7° C./minute; holding at 55° C. for about 30 minutes; ramp from 55° C. to 80° C. at a ramp rate of about 7° C./minute; holding at 80° C. for about 30 minutes; ramp from 80° C. to 100° C. at a ramp rate of about 7° C./minute; and holding at 100° C. for about 30 minutes.

After de-molding, cast-molded SiHy contact lenses are extracted with PAA-coating solution for 30 minutes at 40° C. and for 90 min at 40° C. then rinsed in PB (phosphate buffer containing about 0.077 wt. % NaH₂PO₄·H₂O and about 0.31 wt. % Na₂HPO₄·2H₂O) for 15 min. The resultant silicone hydrogel contact lenses are packaged/sealed in polypropylene lens packaging shells (or blisters) (one lens per shell) with 0.65 mL of IPC saline, and then autoclaved (sterilized) at 121° C. for 45 minutes.

### EXAMPLE 5

The Properties of lenses prepared in Examples 3 and 4 are evaluated and the results are reported in Table 5.

TABLE 5
Properties of the lenses

| | Formulation 3-1 | Formulation 3-2 | Formulation 4-1 |
|---|---|---|---|
| Lens Properties | Control with UV28 | with UV35 | with UV35 |
| Curing | Lightstream | | Thermal |
| Water Content | 32.26 ± 0.18 | 32.22 ± 0.41 | 48.61 ± 0.092 |
| Diameter | 14.235 ± 0.03 | 14.083 ± 0.04 | 14.012 ± 0.0172 |
| AC₍ₘₘ₎ | | | |
| IP | 7.7 ± 0.4 | 7.6 ± 0.2 | 7.4 ± 0.5 |
| Dkc (barrer) | 130 ± 6 | 129 ± 4 | 87 ± 4 |
| Modulus (MPa) | 0.64 ± 0.03 | 0.59 ± 0.01 | 0.70 ± 0.021 |
| ETB(%) | 185 ± 18 | 191 ± 12 | 224 ± 12 |
| % T 380-450 nm | 66% | 30% T | 32% T |

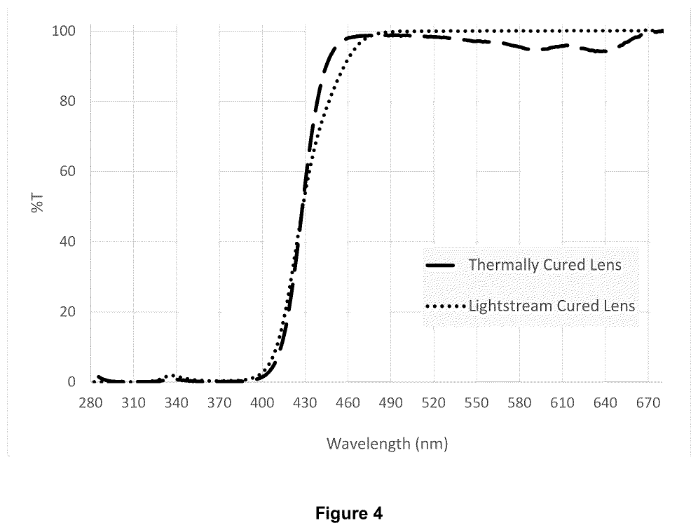

FIG. 4 shows the UV/visible transmission spectra of contact lenses obtained from lens formulations 3-2 and 4-1.

### EXAMPLE 6

This example reports the studies of solubility and stability of UV35.

### Preparation of Lens Formulations

Four lens formulations are prepared to have the compositions shown in Tables 6 and 7.

TABLE 6

| | Lens formulation (wt %) | |
|---|---|---|
| | 6-1 | 6-2 |
| Components | Control with UV28 | With UV35 |
| H-TEMPO | 0.04 | 0.02 |
| DMA | 23.13 | 23.00 |
| 1-PrOH | 22.42 | 21.51 |
| UV35 | 0 | 2.11 |
| UV28 | 1.00 | 0 |
| Norbloc | 0.50 | 0 |
| DMPC | 0.76 | 0.75 |
| L-PEG2000 | 0.61 | 0.61 |
| TRIS-Am | 19.86 | 20.15 |
| CE-PDMS | 31.68 | 31.50 |
| TEB-Ge | 0 | 0 |
| BMBDE-Ge | 0 | 0 |

TABLE 7

| | 6-3 | 6-4 |
|---|---|---|
| Components | Control with UV28 | with UV35 |
| mPDMS | 33.10 | 33.13 |
| G1 | 5.84 | 5.84 |
| N-Vinyl-2-Pyrrolidone (NVP) | 38.92 | 38.96 |
| Methyl Methacrylate (MMA) | 8.76 | 8.77 |
| Ethylene glycol methyl ether | 9.93 | 9.94 |
| methacrylate( EGMA) | | |
| Trieethylene glycol dimethacrylate | 0.29 | 0.29 |
| (TEGMA) | | |
| Allyl Methacrylate (AMA) | 0.097 | 0.97 |
| Norbloc | 0 | 0 |
| VAZO 64 | 0 | 0 |
| RB247 | 0 | 0 |
| 2-Methyl-2-Butanol | 0.97 | 0.96 |
| UV35 | 0 | 2.00 |
| UV28 | 2.10 | 0 |

All formulations in the amount of 10 g are mixed at room temperature for 1-2 hours. Following initial mixing, Lightstream formulations (6-1 and 6-2) are mixed for an additional 1-2 hours at 40° C. UV28 in Lightstream Control formulation (6-1) does not dissolve and this formulation is not subjected to stability study. In the contrary, UV35 dissolves completely in Lightstream formulation 6-2 upon stirring at 40° C. for 30 min. Both benzotriazoles-UV28 and UV35 dissolve in thermally cured formulations (6-3 and 6-4). Formulations are subjected to storage stability study and results are presented in the Table 8. Room temperature and refrigerated (3° C.) conditions are tested.

TABLE 8

| Storage | 6-1 (Control) | 6-2 | 6-3 (Control) | 6-4 |
|---|---|---|---|---|
| Conditions | with UV28 | With UV35 | with UV28 | with UV35 |
| Curing | Lightstream | | Thermal | |
| Benzotriazole | 1.01 | 2.1 | 2.0 | 2.1 |
| Loading (wt %) | | | | |
| % T 380-450 nm | n/a¹ | 30% T | 27% T | 27% T |
| Room Temp. | n/a¹ | Stable³ | Precipitated² | Stable³ |
| (25° C.) | | | | |
| Refrigeration | n/a¹ | n/a⁴ | Precipitated² | Stable³ |
| (3° C.) | | | | |

¹Benzotriazole (UV28) is not soluble in the formulation, UV-VIS spectrum is not recorded and sample is not subjected to stability study;
²Precipitated after overnight storage;
³Stable over the course of 14 days, no precipitation;
⁴Lightstream formulation is stored at room temperature in amber container to protect from light (at refrigerated conditions DMPC precipitates very quickly).

### EXAMPLE 7

Four HEVL-absorbing vinylic monomers listed in Table 9 are used in this example. They can be represented by the following structural formula of

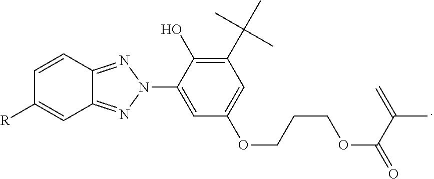

TABLE 9

| | UV29 | UV28 | UV40 | UV23 |
|---|---|---|---|---|
| R group | —H | —Cl | —C(O)OCH₃ | —CF₃ |
| σₚₐᵣₐ | 0.00 | 0.23 | 0.45 | 0.54 |
| Absorption Peak (nm) | 355 | 364 | 377 | 368 |

Table 9 shows that one can shift the HEVL-absorption peak of a HEVL-absorbing vinylic monomer to a longer wavelength by changing the electron withdrawing or donating ability of the substituent R on the aromatic ring.

It is reported that effects of a change in the electron withdrawing or donating capability of the substituent on the absorption peak can be represented using linear free energy relationships, specifically the Hammett Equation (Jaffe, H.H. Chem. Rev., 1953, 53, 191).

Table 9 shows the correlation between the empirical substituent parameter (σₚₐᵣₐ) and the shift in the absorbance peak.

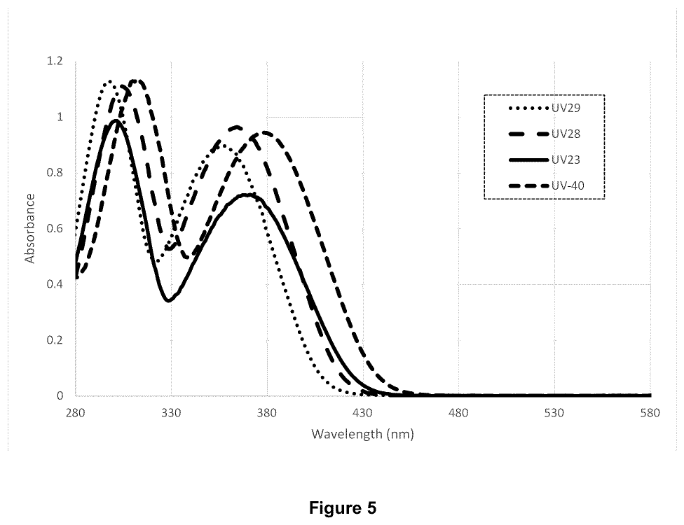

FIG. 5 shows UV-VIS absorption spectra of dilute solutions of 4 HEVL-absorbing benzotriazole-containing vinylic monomers in 1-propanol.

In general, the more the electron withdrawing character, the more the red shift. In case of UV40 (R═—C(O)OCH₃) there might be some other factors that are responsible for significantly higher than expected red shift based on the values of the empirical substituent parameter (σₚₐᵣₐ).

All the publications and patents which have been cited herein above are hereby incorporated by reference in their entireties.

What is claimed is:

1. A HEVL-absorbing benzotriazole vinylic monomer of any one of formula (I) or (II) 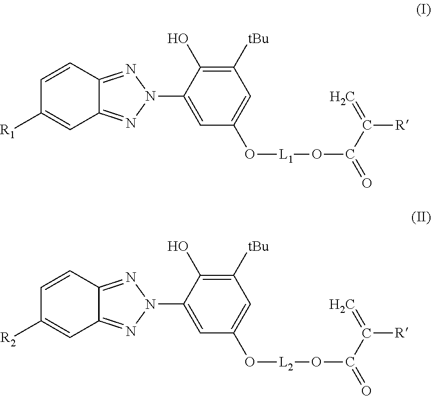
in which: tBu is tert-butyl group, R′ is H or CH₃, R₁ is Cl or CF₃, L₁ is a divalent C₅-C₆ alkylene group or a divalent group of —C₂H₄—(OC₂H₄)ₙ—, R₂ is an organic group which is free of any halogen atom and has a Hammett substituent constant (σₚ) of from about 0.15to about 0.70, L₂ is a divalent C₂-C₁₀ alkylene group or a divalent group of —C₂H₄—(OC₂H₄)ₙ—, n is 1, 2 or 3.

2. The HEVL-absorbing benzotriazole vinylic monomer of claim 1, being of formula (I).

3. The HEVL-absorbing benzotriazole vinylic monomer of claim 1, being of formula (II).

4. The HEVL-absorbing benzotriazole vinylic monomer of claim 3, wherein R₂ has a Hammett substituent constant (σₚ) of from about 0.20 to about 0.65.

5. The HEVL-absorbing benzotriazole vinylic monomer of claim 4, wherein R₂ is —CH₂CN, —OCOCH₃, —CO(CH₃)₃, —SO₃, —CONH₂, —OSO₂CH₃, —CONHCH₃, —CON(CH₃)₂, —CH₂N⁺(CH₃)₃, —COOH, —COOCH₃, —COOC₂H₅, —COC₂H₅, —COCH₃, or —CN.

6. A method for making HEVL-filtering hydrogel contact lenses, comprising the steps of:
(1) obtaining a polymerizable composition comprising
(a) from about 0.1% to about 2.5% by weight of at least one UV-absorbing vinylic monomer,
(b) from about 0.2% to about 3.0% by weight of at least one HEVL-absorbing benzotriazole vinylic monomer of any one of formula (I) or (II) 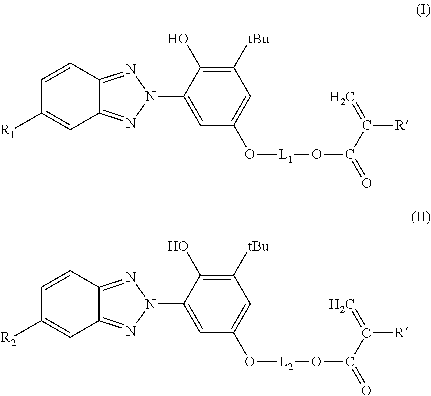
in which: tBu is tert-butyl group, R′ is H or CH₃, R₁ is Cl or CF₃, L₁ is a divalent C₅-C₆ alkylene group or a divalent group of —C₂H₄—(OC₂H₄)ₙ—, R₂ is an organic group which is free of any halogen atom and has a Hammett substituent constant (σₚ) of from about 0.15 to about 0.70 (preferably from about 0.20 to about 0.65), L₂ is a divalent C₂-C₁₀ alkylene group or a divalent group of —C₂H₄—(OC₂H₄)ₙ—, n is 1, 2 or 3,
(c) from about 0.1% to about 2.0% by weight of at least free-radical initiator,
(d) at least one blue-tinting agent which is Cu(II)-phthalocyanine blue pigment particles or at least one polymerizable blue dye, and
(e) at least one polymerizable component selected from the group consisting of a hydrophilic vinylic monomer, a siloxane-containing vinylic monomer, a polysiloxane vinylic crosslinker, a non-silicone vinylic crosslinker, a non-silicone hydrophobic vinylic monomer, and combinations thereof;
(2) introducing the polymerizable composition into a mold for making a hydrogel contact lens, wherein the mold has a first mold half with a first molding surface defining the anterior surface of the contact lens and a second mold half with a second molding surface defining the posterior surface of the contact lens, wherein said first and second mold halves are configured to receive each other such that a cavity is formed between said first and second molding surfaces; and
(3) curing thermally or actinically the lens formulation in the mold to form the HEVL-filtering hydrogel contact lens, wherein the HEVL-filtering hydrogel contact lens has a UVA % T of less than 5%, UVB%T of about 1% or less, a HEVL % T of about 35% or less.

7. The method of claim 6, wherein the free-radical initiator is a thermal initiator, wherein the step of curing is carried out thermally.

8. The method of claim 6, wherein the free-radical initiator is a photoinitiator, wherein the step of curing is carried out by irradiation with a visible light having a wavelength within the range from 450 nm to 510 nm.

9. The method of claim 8, wherein the photoinitiator is a benzoylphosphine oxide photoinitiator, an acyl germanium photoinitiator, an acyltin photoinitiator, or combinations thereof.

10. The method of claim 8, wherein the photoinitiator is an acyl germanium photoinitiator.

11. A HEVL-filtering hydrogel contact lens, comprising a crosslinked polymeric material which comprises:
(1) repeating units of
(a) at least one UV-absorbing vinylic monomer,
(b) at least one HEVL-absorbing benzotriazole vinylic monomer of any one of formula (I) or (II) 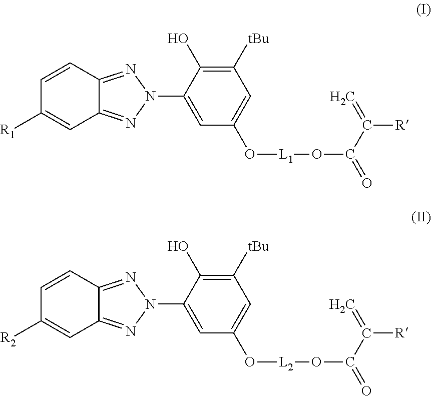
in which: tBu is tert-butyl group, R′ is H or CH₃, R₁ is Cl or CF₃, L₁ is a divalent C₅-C₆ alkylene group or a divalent group of —C₂H₄—(OC₂H₄)ₙ—, R₂ is an organic group which is free of any halogen atom and has a Hammett substituent constant (σₚ) of from about 0.15 to about 0.70 (preferably from about 0.20 to about 0.65), L₂ is a divalent C₂-C₁₀ alkylene group or a divalent group of —C₂H₄—(OC₂H₄)ₙ—, n is 1, 2 or 3, and
(c) at least one polymerizable component selected from the group consisting of a hydrophilic vinylic monomer, a siloxane-containing vinylic monomer, a polysiloxane vinylic crosslinker, a non-silicone vinylic crosslinker, a non-silicone hydrophobic vinylic monomer, and combinations thereof; and
(2) Cu(II)-phthalocyanine blue pigment particles distributed therein and/or repeating units of a polymerizable blue dye,
wherein the HEVL-filtering hydrogel contact lens has a UVA % T of less than 5%, UVB%T of about 1% or less, a HEVL % T of about 35% or less, and a color expressed with a*≤−5 and b*≤+18.

12. The HEVL-filtering hydrogel contact lens of claim 11, wherein the crosslinked polymeric material comprises at least one polymerizable blue dye which is 1,4-bis((2-methoxyethyl)amino)-anthraquinone and/or 1,4-bis(4-(2-methacryloxyethyl)phenylamino)-anthraquinone.

13. The HEVL-filtering hydrogel contact lens of claim 11, wherein the crosslinked polymeric material comprises Cu(II)-phthalocyanine blue pigment particles distributed therein.

14. The HEVL-filtering hydrogel contact lens of claim 11, wherein said at least one UV-absorbing vinylic monomer comprises 2-(2′-hydroxy-5′-vinylphenyl)-2H-benzotriazole, 2-(2′-hydroxy-5′-methacryloxyphenyl)-2H-benzotriazole, 2-(2′-hydroxy-5′-acryloyloxyphenyl)-2H-benzotriazole, 2-[2′-hydroxy-5′-(2-methacryloxyethyl)phenyl)]-2H-benzotriazole (Norbloc), 2-[2′-hydroxy-5′-(2-acryloxyethyl)phenyl)]-2H-benzotriazole, 2-(2′-hydroxy-5′-methacryloxypropyl-phenyl)-2H-benzotriazole, 2-(2′-hydroxy-5′-acryloxypropylphenyl)-2H-benzotriazole, or combinations thereof.

15. The HEVL-filtering hydrogel contact lens of claim 14, wherein said at least one UV-absorbing vinylic monomer is 2-[2′-hydroxy-5′-(2-methacryloxyethyl)phenyl)]-2H-benzotriazole.

16. The HEVL-filtering hydrogel contact lens of claim 14, wherein said at least one HEVL-absorbing benzotriazole vinylic monomer comprises a HEVL-absorbing benzotriazole vinylic monomer of formula (I).

17. The HEVL-filtering hydrogel contact lens of claim 14, wherein said at least one HEVL-absorbing benzotriazole vinylic monomer comprises a HEVL-absorbing benzotriazole vinylic monomer of formula (II).

18. The HEVL-filtering hydrogel contact lens of claim 14, wherein the crosslinked polymeric material comprises repeating units of at least one hydrophilic vinylic monomer selected from the group consisting of N,N-dimethyl (meth)acrylamide, 2-acrylamidoglycolic acid, N-hydroxypropyl (meth)acrylamide, N-hydroxyethyl (meth)acrylamide, N-[tris(hydroxymethyl)-methyl] (meth)acrylamide, N-vinylpyrrolidone, N-vinyl formamide, N-vinyl acetamide, N-vinyl isopropylamide, N-vinyl-N-methyl acetamide, N-methyl-3-methylene-2-pyrrolidone, 1-methyl-5-methylene-2-pyrrolidone, 5-methyl-3-methylene-2-pyrrolidone, 2-hydroxyethyl (meth)acrylate, hydroxypropyl (meth)acrylate, methoxyethyl (meth)acrylate, trimethylammonium 2-hydroxy propylmethacrylate hydrochloride, aminopropyl methacrylate hydrochloride, dimethylaminoethyl methacrylate (DMAEMA), glycerol methacrylate (GMA), a C₁-C₄-alkoxy polyethylene glycol (meth)acrylate having a weight average molecular weight of up to 1500, polyethylene glycol (meth)acrylate having a weight average molecular weight of up to 1500, methacrylic acid, acrylic acid, methacryloxyethyl phosphocholine, methacryloxypropyl phosphocholine, N-2-hydroxyethyl vinyl carbamate, N-carboxyvinyl-β-alanine, N-carboxyvinyl-α-alanine, and mixtures thereof.

19. The HEVL-filtering hydrogel contact lens of claim 18, wherein the crosslinked polymeric material is a silicone hydrogel material that comprises repeating units of at least one siloxane-containing vinylic monomer and/or at least one polysiloxane vinylic crosslinker.
